(19) United States
(12) Patent Application Publication
(10) Pub. No.: US 2018/0220988 A1
(43) Pub. Date: Aug. 9, 2018

(54) X-RAY DETECTOR

(71) Applicant: SAMSUNG ELECTRONICS CO., LTD., Suwon-si (KR)

(72) Inventor: Hak-Soo JEON, Suwon-si (KR)

(73) Assignee: SAMSUNG ELECTRONICS CO., LTD., Suwon-si (KR)

(21) Appl. No.: 15/867,134

(22) Filed: Jan. 10, 2018

(30) Foreign Application Priority Data
Feb. 3, 2017 (KR) 10-2017-0015702

(51) Int. Cl. A61B 6/00; H02J 7/02; H02J 50/12; H02J 50/80; H04B 5/00

(52) U.S. Cl. CPC A61B 6/56; H02J 7/025; H04B 5/0037; H02J 50/80; H02J 50/12

(57) ABSTRACT

An X-ray detector is provided. The X-ray detector includes a housing; a panel positioned inside the housing, and configured to detect an X-ray and convert the detected X-ray into an electrical signal; a controller configured to generate an X-ray image using the electrical signal; and an integrated module including a wireless charging module for wirelessly receiving power from a wireless power transmitter and a Near Field Communication (NFC) module for performing NFC with an external electronic device.

### CROSS-REFERENCE TO RELATED APPLICATION

This application claims priority from Korean Patent Application Serial No. 10-2017-0015702, which was filed in the Korean Intellectual Property Office on Feb. 3, 2017, the disclosure of which is incorporated herein by reference in its entirety.

### BACKGROUND

### 1. Field

The exemplary embodiments relate to an X-ray detector having wireless communication and wireless charging functions.

### 2. Description of the Related Art

An X-ray imaging apparatus can acquire an image of the inside of an object using X-rays. The X-ray imaging apparatus irradiates the object with X-rays and detects X-rays transmitted through the object to acquire the image of the inside of the object. When the object is a patient, the X-ray imaging apparatus can be used for medical diagnosis of, for example, injury or illness of the patient, by viewing the appearance of an image of the inside of the patient.

The X-ray imaging apparatus may include an X-ray source for generating X-rays to irradiate a patient with the generated X-rays and an X-ray detector for detecting X-rays transmitted through the patient. In order to image various parts of the patient, the X-ray source may be provided so as to be movable, and the X-ray detector may be mounted on an imaging table or an imaging stand, or may be used in a portable manner.

Various kinds of X-ray detectors can be used in a number of X-ray imaging rooms. After a conventional X-ray detector is connected to a workstation of the X-ray imaging room by a wired cable, the workstation can control the X-ray detector to manually change setting information of the X-ray detector. At this time, repeatedly performing a process in which the workstation determines whether the X-ray detector is registered and registers the X-ray detector for each of various kinds of X-ray detectors may require a long time and extensive user operation. In particular, such an increase in operation time may cause life-threatening problems in an emergency situation.

### SUMMARY

According to the exemplary embodiments, there is provided an X-ray detector which can conveniently set an operating environment by performing wireless transmission/reception with an X-ray imaging apparatus.

According to the exemplary embodiments, there is provided an X-ray detector which is equipped with a wireless charging function and can charge a battery of the X-ray detector without using a separate wired cable.

According to the exemplary embodiments, there is provided an X-ray detector in which an antenna for wireless transmission/reception and a coil for wireless charging are manufactured as a single module, thereby preventing an increase in an installation space inside the X-ray detector.

According to an aspect of an exemplary embodiment, there is provided an X-ray detector including: a housing; a panel positioned inside the housing, and configured to detect an X-ray and convert the detected X-ray into an electrical signal; a controller configured to generate an X-ray image using the electrical signal; and an integrated module including a wireless charging module for wirelessly receiving power from a wireless power transmitter and a Near Field Communication (NFC) module for performing NFC with an external electronic device.

The integrated module may be configured to, when an NFC start command is acquired while the power is wirelessly received from the wireless power transmitter, stop reception of the power before performing the NFC, and turn on the NFC module.

The wireless charging module may be configured to wirelessly receive the power based on a Wireless Power Consortium (WPC) standard, and request stoppage of transmission of the power from the wireless power transmitter based on an ON/OFF keying modulation/demodulation scheme defined in the WPC standard.

The wireless charging module may be configured to request the stoppage of transmission of the power from the wireless power transmitter, using an end power transfer packet defined in the WPC standard.

The wireless charging module may be configured to wirelessly receive the power based on an Alliance for Wireless Power (A4WP) standard, and the wireless charging module may include a Bluetooth Low Energy (BLE) communication module that complies with the A4WP standard.

The wireless charging module may be configured to transmit a message for requesting stoppage of transmission of the power from the wireless power transmitter, through the BLE communication module.

The message for requesting the stoppage of transmission of the power may be a PRU dynamic message defined in the A4WP standard.

The integrated module may be configured to: transmit a signal including identification information of the X-ray detector through the NFC module, turn off the NFC module when the signal including the identification information of the X-ray detector is completely transmitted, and request transmission restart of the power from the wireless power transmitter.

The integrated module may be configured to, when an imaging start command for the X-ray is acquired using the panel while the power is wirelessly received from the wireless power transmitter, request stoppage of reception of the power before performing the NFC, and turn on the NFC module.

The integrated module may be configured to, when imaging for the X-ray is completed using the panel, request restart of power transmission from the wireless power transmitter.

The wireless charging module may include a coil for receiving the power from the wireless power transmitter, the NFC module includes an antenna for transmitting and receiving a signal by the NFC, and the antenna is disposed to surround the coil.

The X-ray detector may further include a frame configured to be disposed between the panel and the integrated module.

The X-ray detector may further include at least one circuit board configured to be electrically connected to the panel and a battery of the X-ray detector, wherein the wireless charging module is disposed to be spaced apart from the at least one circuit board so as to avoid electromagnetic interference.

The battery may be disposed between the wireless charging module and the at least one circuit board.

The X-ray detector may further include a blocking member configured to be disposed between the at least one circuit board and the wireless charging module such that the blocking member blocks electromagnetism generated from the wireless charging module.

According to another aspect of an exemplary embodiment, there is provided an X-ray detector including: a housing; a panel positioned inside the housing, and configured to detect an X-ray and convert the detected X-ray into an electrical signal; a controller configured to generate an X-ray image using the electrical signal; and an integrated module configured to include a wireless charging module for wirelessly receiving power from a wireless power transmitter and a Bluetooth Low Energy (BLE) communication module for performing BLE communication with the wireless power transmitter, wherein, when an imaging start command for the X-ray is acquired using the panel while the power is wirelessly received from the wireless power transmitter, the integrated module requests stoppage of transmission of the power from the wireless power transmitter.

The BLE communication module may be configured to transmit a message for requesting the stoppage of transmission of the power from the wireless power transmitter.

The message for requesting the stoppage of transmission of the power is a PRU dynamic message defined in an Alliance for Wireless Power (A4WP) standard.

The integrated module may be configured to, when imaging for the X-ray is completed using the panel, request restart of power transmission from the wireless power transmitter.

According to yet another aspect of an exemplary embodiment, there is provided an X-ray imaging apparatus including: an X-ray output device configured to apply an X-ray; an X-ray detector including a sensing unit for detecting the X-ray, a battery, a wireless charging module electrically connected to the battery to generate current using a change in an external magnetic field, and an Near Field Communication (NFC) module for performing NFC with an external electronic device; and a reception portion configured to receive the X-ray detector.

According to an X-ray detector according to an exemplary embodiment, environment setting with a plurality of X-ray imaging apparatuses may be carried out using an antenna, whereby it is possible to reduce the time and effort required for a user to manually operate the environment setting of the X-ray detector.

According to an X-ray detector according to an exemplary embodiment, an antenna for wireless transmission/reception may be arranged to surround a coil for wireless charging, whereby it is possible to prevent an increase in the installation space of the X-ray detector due to the arrangement of the antenna and the coil.

According to an X-ray detector according to an exemplary embodiment, a user may more conveniently charge a battery of the X-ray detector by charging the battery through wireless charging, and a terminal connected to a separate wired cable may not be needed, whereby it is possible to prevent foreign matter (e.g., blood of a patient or dust) from being introduced into the inside of the X-ray detector.

### BRIEF DESCRIPTION OF THE DRAWINGS

The above and other aspects, features, and advantages of the exemplary embodiments will be more apparent from the following detailed description taken in conjunction with the accompanying drawings, in which:

FIG. 1A is a plan view showing an X-ray detector according to an exemplary embodiment;

FIG. 1B is a plan view showing an X-ray detector according to an exemplary embodiment;

FIG. 2A is a plan view showing an internal view of an X-ray detector according to an exemplary embodiment;

FIG. 2B is a cross-sectional view showing an X-ray detector according to an exemplary embodiment;

FIG. 3A is a perspective view showing an X-ray imaging room to which an X-ray detector according to an exemplary embodiment;

FIG. 3B is a block diagram showing an X-ray detector and a workstation according to an exemplary embodiment;

FIGS. 4A to 4I are conceptual diagrams illustrating an X-ray detector according to various exemplary embodiments;

FIG. 5A is a flowchart illustrating the operation of a wireless power transmitter and an X-ray detector according to an exemplary embodiment;

FIG. 5B is a flowchart illustrating the operation of a wireless power transmitter and an X-ray detector according to another exemplary embodiment;

FIG. 6 is a flowchart illustrating the operation of a wireless power transmitter and an X-ray detector according to yet another exemplary embodiment;

FIG. 7 is a flowchart illustrating the operation of an X-ray detector according to an exemplary embodiment;

FIGS. 8A to 8C are conceptual diagrams illustrating a communication method in a WPC standard according to an exemplary embodiment;

FIGS. 9A and 9B are block diagrams illustrating an X-ray detector and a wireless power transmitter which comply with an alliance for wireless power (A4WP) standard, according to an exemplary embodiment;

FIG. 10 is a signal flow diagram illustrating the operation of a wireless power transmitter and a wireless power receiver included in an X-ray detector in accordance with an A4WP standard, according to an exemplary embodiment;

FIG. 11A is a block diagram illustrating an X-ray detector according to an exemplary embodiment;

FIG. 11B is a block diagram illustrating an X-ray detector according to another exemplary embodiment; and

FIG. 12 is a circuit diagram illustrating an X-ray detector according to yet another exemplary embodiment.

### DETAILED DESCRIPTION

Hereinafter, various exemplary embodiments of the present disclosure will be described with reference to the accompanying drawings. The exemplary embodiments and the terms used therein are not intended to limit the technology disclosed herein to specific forms, and should be understood to include various modifications, equivalents, and/or alternatives to the corresponding exemplary embodiments. In describing the drawings, similar reference numerals may be used to designate similar constituent elements. A singular expression may include a plural expression unless they are definitely different in a context. As used herein, singular forms may include plural forms as well unless the context clearly indicates otherwise. The expression “a first”, “a second”, “the first”, or “the second” used in various exemplary embodiments of the present disclosure may modify various components regardless of the order and/or the importance but does not limit the corresponding components. When an element (e.g., first element) is referred to as being “(functionally or communicatively) connected,” or “directly coupled” to another element (second element), the element may be connected directly to the another element or connected to the another element through yet another element (e.g., third element).

The expression “configured to” as used in various exemplary embodiments of the present disclosure may be interchangeably used with, for example, “suitable for”, “having the capacity to”, “designed to”, “adapted to”, “made to”, or “capable of” in terms of hardware or software, according to circumstances. Alternatively, in some situations, the expression “device configured to” may mean that the device, together with other devices or components, “is able to”. For example, the phrase “processor adapted (or configured) to perform A, B, and C” may mean a dedicated processor (e.g., embedded processor) only for performing the corresponding operations or a generic-purpose processor (e.g., Central Processing Unit (CPU) or Application Processor (AP)) that can perform the corresponding operations by executing one or more software programs stored in a memory device.

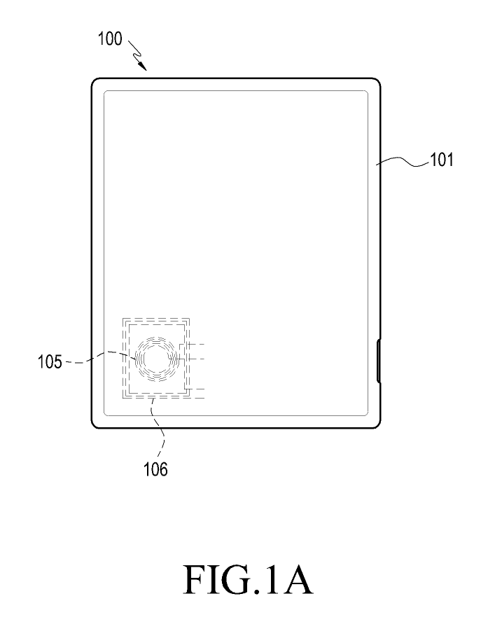

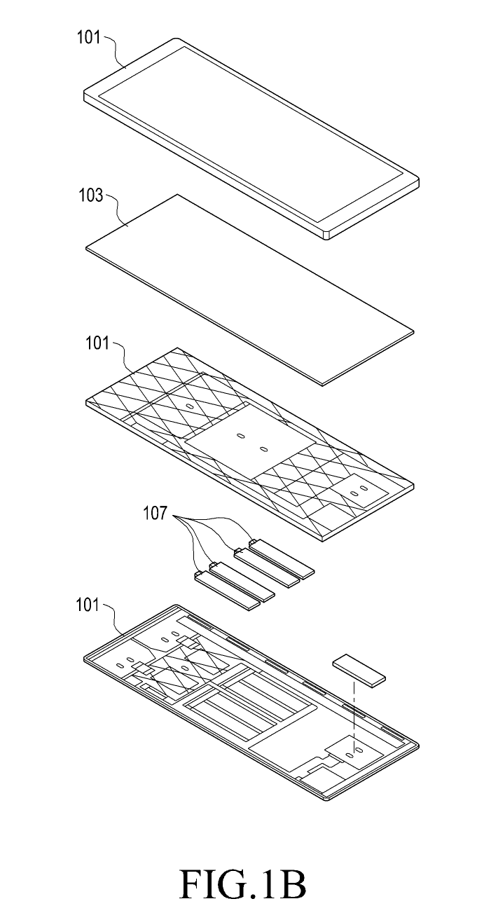

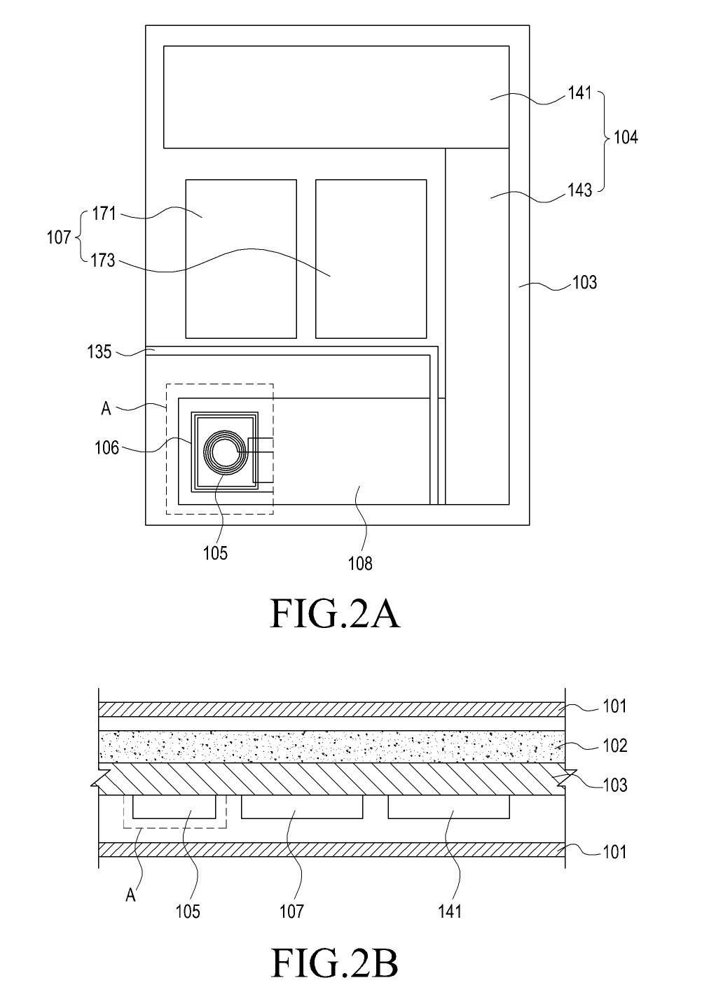

FIG. 1A is a plan view showing an X-ray detector according to an exemplary embodiment, FIG. 1B is a exploded view of an X-ray detector according to an exemplary embodiment, FIG. 2A is a plan view showing an internal view of an X-ray detector according to an exemplary embodiment, and FIG. 2B is a cross-sectional view showing an X-ray detector according to an exemplary embodiment.

Referring to FIGS. 1A to 2B, an X-ray detector 100 according to an exemplary embodiment may include a housing 101, a frame 103, a sensing unit 102, at least one circuit board 104, a first wireless charging coil 105, a first antenna 106, and a battery 107. The first wireless charging coil 105 and the first antenna 106 may be integrated into a module A.

The housing 101 may protect the inside of the X-ray detector 100 while forming the appearance thereof. The housing 101 may be made of a corrosion-resistant material. For example, the housing 101 may be made of a material which is not easily corroded by the blood of a user or the like.

The frame 103 may be disposed inside the housing 101.

The sensing unit 102 may be disposed on a first surface of the frame 103, and may detect X-rays. For example, the sensing unit 102 may include a panel including a plurality of pixels capable of detecting received X-rays. For example, the sensing unit 102 may include a gate driver for determining a row to be scanned of the plurality of pixels and a Read Out Integrated Circuit (ROIC) for reading out at least one of the plurality of pixels. Each of the plurality of pixels may include a photoelectric conversion element for converting the received X-rays into electrical signals. The sensing unit 102 will be described later in more detail.

The at least one circuit board 104 may be disposed on a second surface of the frame 103. The circuit board 104 may include a first circuit board 141 and a second circuit board 143. A controller that is electrically connected to the sensing unit 102 and a memory that stores an electrical signal transmitted from the sensing unit 102 may be mounted on the first circuit board 141. The first circuit board 141 may be disposed adjacent to one side surface of the frame 103. The second circuit board 143 may be electrically connected to the first circuit board 141 and may be disposed adjacent to the other side surface of the frame 103.

The first wireless charging coil 105 may be disposed on one surface of a substrate and may generate a current or a voltage using a change in an external magnetic field, and thereby may receive power wirelessly. The first wireless charging coil 105 may be electrically connected to the battery 107 so that the current generated by the first wireless charging coil 105 may charge the battery 107. The X-ray detector 100 according to exemplary embodiments may further include at least one element for processing the power received from the first wireless charging coil 105. For example, the X-ray detector 100 may further include a rectifier for rectifying the power of an AC waveform received from the first wireless charging coil 105 into power of a DC waveform, a converter for converting the rectified power into a voltage suitable for a charger, and the charger for receiving the converted power and charging the battery using a predefined charging scheme (e.g., a constant-voltage (CV) charging scheme, a constant-current (CC) charging scheme, or a rapid charging scheme). Alternatively, although not shown, the power received from the first wireless charging coil 105 may be transmitted to a power management integrated circuit (PMIC) rather than to the charger. In this case, the PMIC may process and supply the received power so as to be suitable for a variety of hardware in the X-ray detector 100.

The first wireless charging coil 105 may be wound in a spiral shape, but those skilled in the art will readily understand that there is no limitation as to the winding form or the number of windings of the first wireless charging coil. The first wireless charging coil 105 may be implemented so as to conform to a wireless charging standard. For example, when the X-ray detector 100 complies with a wireless power consortium (WPC) standard (or a Qi standard), the first wireless charging coil 105 may be implemented to operate at, for example, 100 to 205 kHz. For example, when the X-ray detector 100 complies with an alliance for wireless power (A4WP) standard (or an air fuel alliance (AFA) standard), the first wireless charging coil 105 may be implemented to operate at, for example, 6.78 MHz. Alternatively, when the X-ray detector 100 is based on a remote transmission scheme (e.g., an RF scheme), the first wireless charging coil 105 may be implemented to operate at, for example, 5.8 GHz. Meanwhile, it will be appreciated by those skilled in the art that the above-described numerical values are merely examples and that there is no limitation on the implementation form or element value of the first wireless charging coil 105 as long as the first wireless charging coil 105 receives power wirelessly from an external wireless power transmitter.

The first antenna 106 may surround the first wireless charging coil 105. The first antenna 106 may receive current from the battery 107 to transmit and receive a wireless signal. Alternatively, the first wireless charging coil 105 may be implemented to surround the first antenna 106. Arrangement of the first wireless charging coil 105 and the first antenna 106 is not limited thereto and may vary according to an exemplary embodiment.

The first wireless charging coil 105 may be disposed to be spaced apart from the circuit board 104 so as not to have an electromagnetic influence on the circuit board 104. For example, the first wireless charging coil 105 may be disposed to be spaced as far as possible from the circuit board 104 on the first surface of the frame 103.

The battery 107 may be disposed between the first circuit board 141 and the first wireless charging coil 105. The battery 107 may be made of a conductive material (e.g., metal) to prevent electromagnetic waves generated from the first wireless charging coil 105 from being transmitted to the first circuit board 141.

The X-ray detector 100 may further include a blocking member 108. The blocking member 108 may be disposed between the circuit board 104 and the first wireless charging coil 105. The blocking member 108 may be made of a conductive material (e.g., metal) to prevent electromagnetic waves generated from the first wireless charging coil 105 from being transmitted to the circuit board 104.

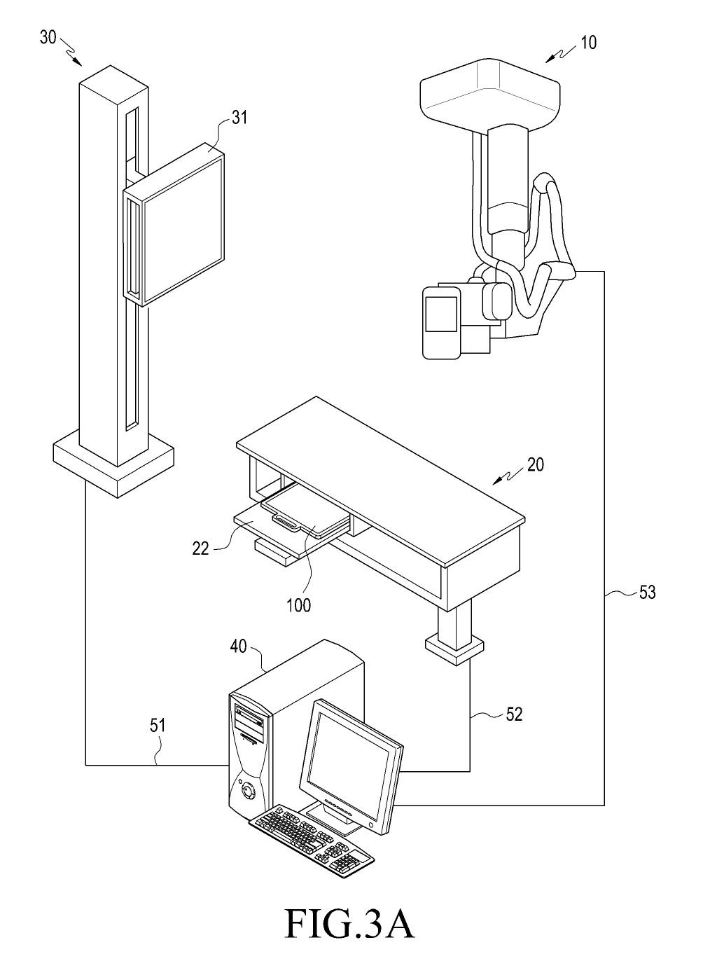

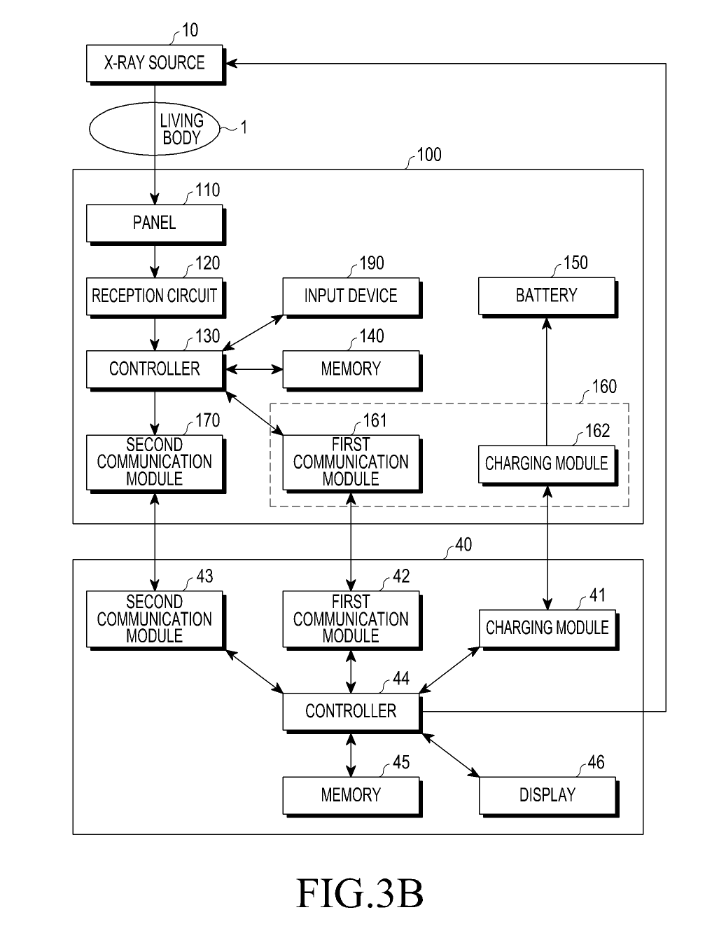

FIG. 3A is a perspective view showing an X-ray imaging room to which an X-ray detector according to an exemplary embodiment is applied.

Referring to FIG. 3A, an X-ray source 10, an imaging table 20, an imaging stand 30, a workstation 40, and an X-ray detector 100 may be located in the X-ray imaging room.

The X-ray source 10 is a device for irradiating an object (e.g., a living body) with X-rays. Here, the object may be a human or an animal, but is not limited thereto. That is, the object may be anything that can be transmitted by X-rays.

The imaging table 20 may include an upper plate on which an object (e.g., a patient) can be placed and a reception portion 22 for receiving the X-ray detector 100. The reception portion 22 may be drawn out of or inserted into the imaging table 20 through a guide portion (not shown). The imaging table 20 will be described in more detail later with reference to the drawings.

The imaging stand 30 may include a reception portion 31 for receiving the X-ray detector 100. The reception portion 31 may be moved along the longitudinal direction of the imaging stand 30 through a guide portion. The imaging stand 30 will be described in more detail later with reference to the drawings.

The workstation 40 may perform communications 51, 52, and 53 with at least one of the X-ray source 10, the imaging table 20, and the imaging stand 30 in a wired or wireless manner. For example, the workstation 40 may transmit an imaging command signal to the X-ray source 10, and the X-ray source 10 may apply X-rays accordingly. The imaging command signal may further include information such as an irradiation amount, an irradiation time, or the like. The workstation 40 may transmit a physical movement command signal of the X-ray source 10, and the X-ray source 10 may physically move accordingly. The workstation 40 may transmit a signal for controlling wireless charging to the imaging table 20 or the imaging stand 30. For example, the workstation 40 may transmit the signal for controlling wireless charging to the imaging table 20 or the imaging stand 30 to thereby cause a wireless charging module included in the imaging table 20 or the imaging stand 30 to start or stop charging. Accordingly, a user may operate the workstation 40 to start or stop charging the wireless charging module included in the imaging table 20 or the imaging stand 30.

The workstation 40 may perform wireless communication with the X-ray detector 100. In an exemplary embodiment, the workstation 40 may communicate with the X-ray detector 100 via near field communication (NFC). In this case, the workstation 40 may receive identification information of the X-ray detector 100 from the X-ray detector 100, and may register the X-ray detector 100 using the received identification information. The identification information mentioned here may be a format defined by a manufacturer of the X-ray detector 100. In this case, the X-ray detector 100 may transmit identification information defined by the manufacturer, which is different from the identification information defined by NFC, to the workstation 40 using the NFC. Meanwhile, in another exemplary embodiment, the identification information may comply with a format defined by an NFC standard. In this case, the X-ray detector 100 may transmit the identification information defined by NFC to the workstation 40, and the workstation 40 may register the X-ray detector 100 using the received identification information. Meanwhile, the X-ray detector 100 may communicate with the workstation 40 in a manner defined in the wireless charging standard. For example, the X-ray detector 100 may communicate with the workstation 40 using an ON/OFF keying modulation/demodulation scheme based on an in-band communication scheme. Alternatively, the X-ray detector 100 may communicate with the workstation 40 based on an out-band communication scheme (e.g., a Bluetooth low energy (BLE) scheme). Meanwhile, the X-ray detector 100 may communicate with the workstation 40 based on wireless data communication. Examples of the wireless data communication may include, but are not limited to, LTE, LTE-Advanced (LTE-A), code division multiple access (CDMA), wideband CDMA (WCDMA), universal mobile telecommunications system (UMTS), Wireless Broadband (WiBro), Global System for Mobile communications (GSM), wireless fidelity (Wi-Fi).

Meanwhile, in another exemplary embodiment, the imaging table 20 or the imaging stand 30 may communicate with the X-ray detector 100 using NFC. The X-ray detector 100 may transmit identification information to the imaging table 20 or the imaging stand 30, and the imaging table 20 or the imaging stand 30 may perform registration using the received identification information or may transmit the received identification information to the workstation 40.

FIG. 3B is a block diagram showing an X-ray detector 100 and a workstation 40 according to an exemplary embodiment.

The X-ray detector 100 may include a panel 110, a reception circuit 120, a controller 130, a memory 140, a battery 150, a communication and wireless charging integration module 160, a second communication module 170, and an input device 190. The communication and wireless charging integration module 160 may include a first communication module 161 and a charging module 162.

The workstation 40 may include a charging module 41, a first communication module 42, a second communication module 43, a controller 44, a memory 45, and a display 46.

The X-ray source 10 may apply X-rays under the control of, for example, the controller 44 of the workstation 40. Alternatively, the X-ray source 10 may include an input device and may apply X-rays in response to a command received through the input device. The X-rays applied from the X-ray source 10 may be transmitted through a living body 1 and may be incident on the panel 110.

The panel 110 may include a plurality of pixels, and each of the plurality of pixels may include a photoelectric conversion element. Each of the plurality of pixels may convert the received X-rays into electrical signals and output the electrical signals to the reception circuit 120. The photoelectric conversion element may be constituted of a single-type element or a mixed-type element depending on the material construction method. The photoelectric conversion element constituted of the single-type element corresponds to the case where a portion for detecting X-rays to generate electrical signals and a portion for reading and processing electrical signals are formed of a single-material semiconductor or are manufactured through a single process. For example, a charge-coupled device (CCD) or a complementary metal oxide semiconductor (CMOS), which are light-receiving elements, may be used singly. The photoelectric conversion element constituted of the mixed-type element corresponds to the case where the portion for detecting X-rays to generate electrical signals and the portion for reading and processing electrical signals are made of different materials or are manufactured through different processes. For example, when X-rays are detected using a light-receiving element such as a photodiode, a CCD, a CdZnTe, or the like, and electrical signals are read and processed using a CMOS ROIC, the X-rays may be detected using a strip detector and the electrical signals may be read and processed using the CMOS ROIC or using an a-Si or a-Se flat panel system. In addition, the photoelectric conversion element may be divided into a direct conversion method and an indirect conversion method depending on the method in which X-rays are converted into electrical signals. In the direct conversion method, when an X-ray is applied, an electron-hole pair is temporarily generated inside a light-receiving element, and the electron is moved to an anode and the hole is moved to a cathode by an electric field applied to both ends of the light-receiving element. Here, the photoelectric conversion element may convert this movement into an electrical signal. In the direct conversion method, the material used for the light-receiving element may be a-Se, CdZnTe, HgI2, PbI2, or the like. In the indirect conversion method, when an X-ray applied from the X-ray source reacts with a scintillator to emit a photon with a wavelength in a visible light region, the light-receiving element may detect the emitted photon and convert the detected photon into an electrical signal. In the indirect conversion method, the material used for the light-receiving element is a-Si or the like. Examples of the scintillator include a thin film type Gadolinium Oxysulfide (GADOX) scintillator, a micro-columnar or needle-structured CSI (T1), and the like. In addition, the photoelectric conversion element may be divided into a charge integration mode in which a charge is stored for a predetermined time and a signal is acquired from the stored charge and a photon-counting mode in which counting is performed every time a signal is generated by a single X-ray photon, in accordance with a method in which an electrical signal is acquired.

The reception circuit 120 may include a switching element capable of selecting at least one column of a plurality of pixels and at least one analog-to-digital converter (ADC) capable of performing analog-to-digital conversion of the electrical signal. The reception circuit 120 may further include a gate driver capable of selecting a row to be read out from among a plurality of pixels.

The controller 130 may generate an X-ray image using a processed signal provided from the reception circuit 120. The controller 130 may generate the X-ray image based on, for example, a digital value for each pixel. The controller 130 may store the generated X-ray image in the memory 140. Alternatively, the controller 130 may transmit the generated X-ray image to a second communication module 43 of the workstation 40 via the second communication module 170. Here, the second communication module 170 may be a communication module capable of performing communication at a relatively high speed. Meanwhile, in various embodiments of the present disclosure, the controller 130 may transmit the generated X-ray image to a first communication module 42 of the workstation 40 via the first communication module 161. The first communication module 161 may perform, for example, NFC, and in this case, the X-ray detector 100 may not include the second communication module 170.

The controller 130 and/or a controller 44 may include one or more of a central processing unit (CPU), an application processor (AP), and a communication processor (CP). The controller 130 and/or the controller 44 may perform operations or data processing relating to the control and/or communication of at least one other component of, for example, the X-ray detector 100 and the workstation 40. The controller 130 may be referred to as a processor. In another exemplary embodiment, the controller 130 may be implemented in various forms such as a micro controlling unit (MCU), a minicomputer, a microcomputer (MiCOM), a microprocessor unit (MPU), a field-programmable gate array (FPGA), and the like. Those skilled in the art will readily understand that there is no limitation on the controller 130 as long as it is a device or an element capable of executing or calculating program instructions for performing the operations of the X-ray detector 100 and/or the workstation 40.

The battery 150 may provide power required for the operation of the X-ray detector 100. Although not shown, the power from the battery 150 may be provided to a PMIC (not shown), and the PMIC may appropriately process the power provided from the battery 150 for each piece of hardware and provide the processed power. The charging module 162 may wirelessly receive power from the charging module 41 of the workstation 40. The charging module 162 may charge the battery 150 by rectifying and converting the wirelessly received power. Although not shown, the charging module 162 may provide the processed power to a charger (not shown), and the charger may charge the battery 150 by processing the processed power depending on the method of charging the battery 150. Alternatively, the charging module 162 may provide the processed power to the PMIC.

The first communication module 161 may perform communication with the first communication module 42 of the workstation 40 based on, for example, an NFC method. For example, the first communication module 161 may transmit identification information of the X-ray detector 100 to the first communication module 42. Here, the identification information may be in a format defined by the manufacturer of the X-ray detector 100. In this case, the first communication module 161 may first perform NFC pairing with the first communication module 42. The first communication module 161 may perform the NFC pairing by transmitting the identification information based on a format defined by the NFC and may transmit, to the first communication module 42, the identification information based on the format defined by the manufacturer through the NFC pairing link. Alternatively, the identification information may be based on the format defined by the NFC. The controller 44 of the workstation 40 may register the X-ray detector 100 using the received identification information of the X-ray detector 100. The controller 44 may control an irradiation time, an X-ray irradiation amount, and the like of the X-ray source 10 based on registration information, or may manage an X-ray image received at a later time. The controller 44 may store the registration information, the X-ray image, or the like of the X-ray detector 100 in the memory 45, or may control the registration information, the X-ray image, or the like to be displayed on the display 46.

Meanwhile, the controller 44 may control ON/OFF of the charging module 41. For example, the controller 44 may control the charging module 41 to be turned on or off according to a charging start or charging stop input received through an input device (not shown) of the workstation 40. Alternatively, the charging module 41 may be controlled to be turned on or off according to a charging start or charging stop input from the X-ray detector 100. For example, the charging module 162 may wirelessly receive power from the charging module 41 according to a WPC scheme (or a Qi scheme). The charging module 162 may include at least one load and a switch for performing ON/OFF keying modulation/demodulation for the in-band communication scheme. The charging module 162 may transmit a charging start or charging stop command to the charging module 41 through ON/OFF control of the switch. The charging module 41 may perform charging start or charging stop in response to the received charging start or charging stop command. In an exemplary embodiment, the controller 44 may control to transmit the charging stop command when NFC is performed or when X-ray image photography is performed. The controller 44 may control the charging module 162 so that the charging stop command is transmitted in a communication method defined in the WPC standard, or may control the charging stop command to be transmitted through the second communication module 170. In another exemplary embodiment, the controller 130 may transmit the charging stop command using a sound wave generator (not shown). The controller 130 may control the sound wave generator to generate a sound wave corresponding to the charging stop command, in response to detection of a trigger of various charging stop commands. In this case, the workstation 40 may further include a receiver capable of detecting sound waves, and may control the receiver to analyze the detected sound waves to thus stop wireless charging.

The controller 44 may detect an NFC start command and may transmit the charging stop command to the workstation 40 using the NFC start command as a trigger. Alternatively, the controller 44 may detect a photography command and may transmit the charging stop command to the workstation 40 using the photography command as a trigger. Alternatively, the controller 44 may transmit the charging stop command to the workstation 40 based on the charging stop command received through the input device 190. Accordingly, when a user attempts to start the NFC using the X-ray detector 100, operates the X-ray detector 100 to start imaging, or operates the input device 190, the X-ray detector 100 may transmit the charging stop command to the workstation 40.

After the charging of the charging module 41 is stopped, the controller 44 may perform NFC pairing with the X-ray detector 100 using the first communication module 42. Alternatively, after the charging of the charging module 41 is stopped, the controller 44 may control the X-ray source 10 to apply X-rays.

Meanwhile, the imaging table 20 or the imaging stand 30 may also include substantially the same components as the workstation 40. In an exemplary embodiment, the imaging table 20 and/or imaging stand 30 may be implemented to include both the first communication module 42 and the charging module 41 or to include only the charging module 41.

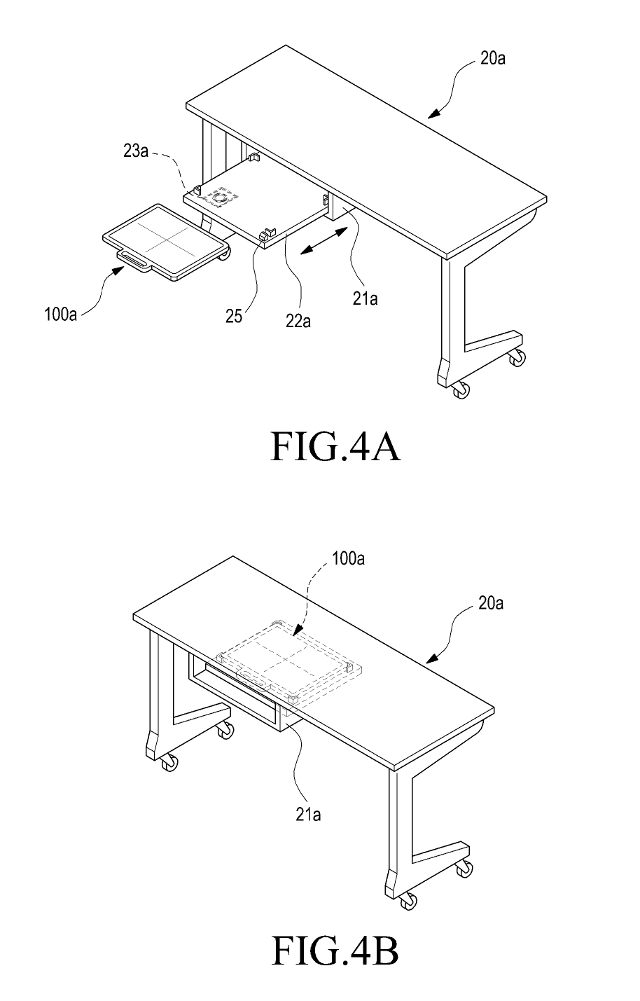

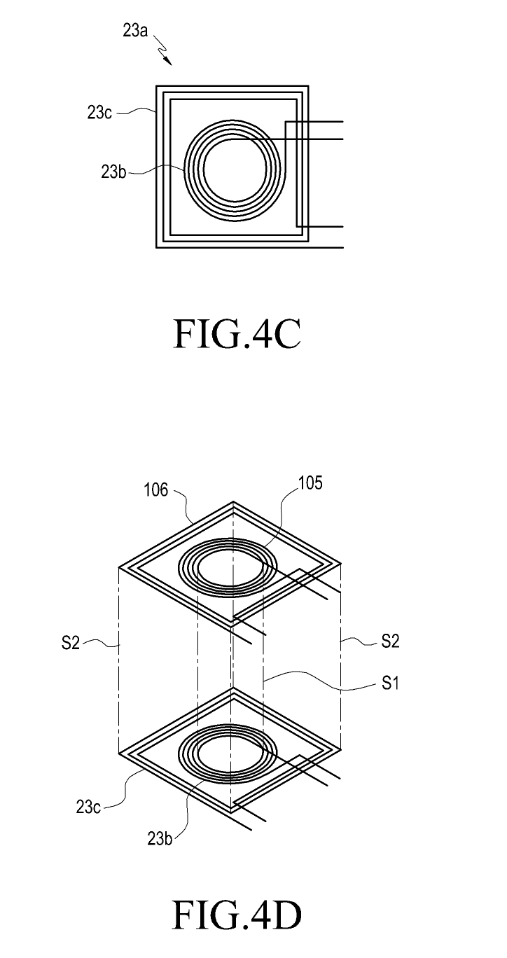

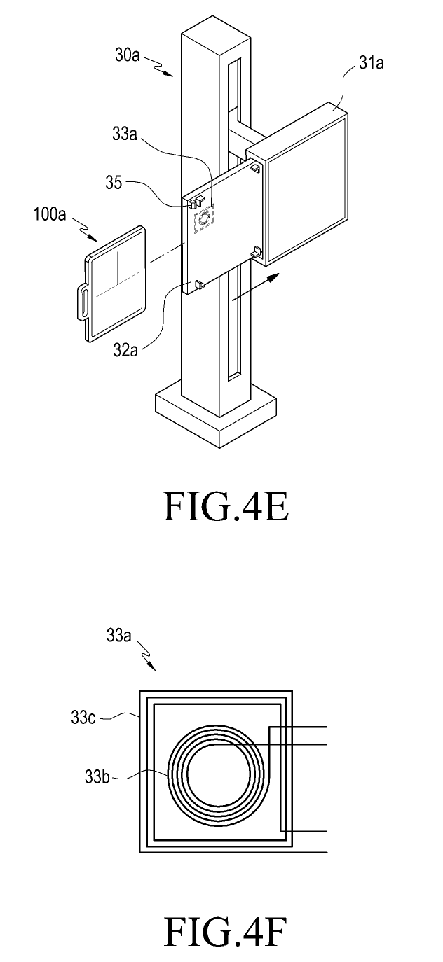

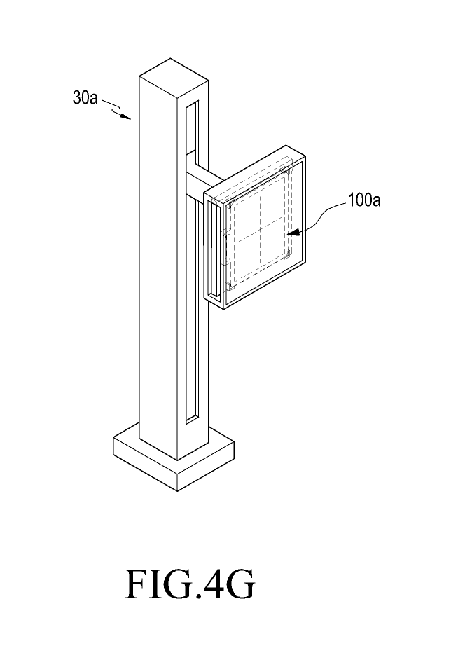

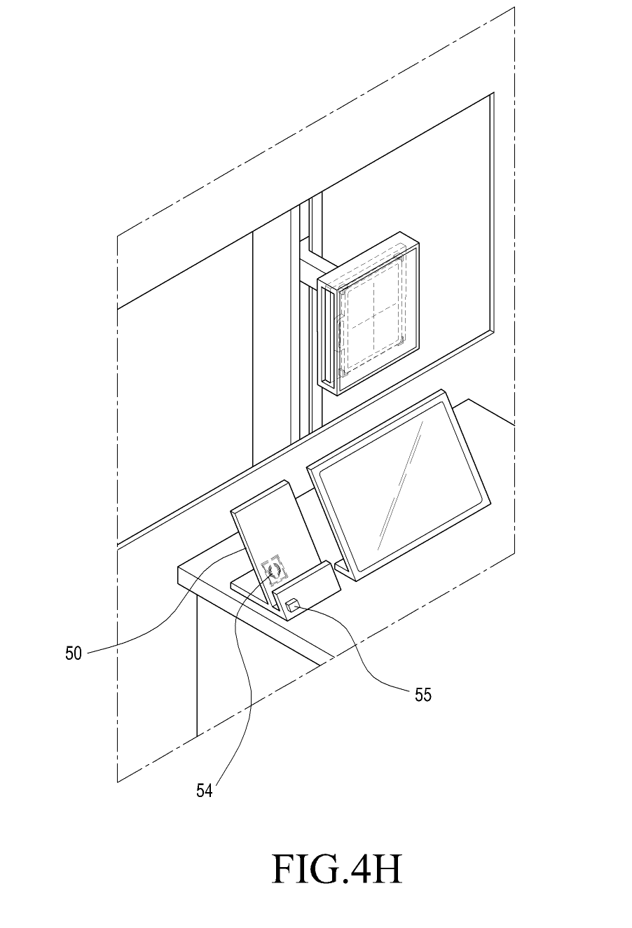

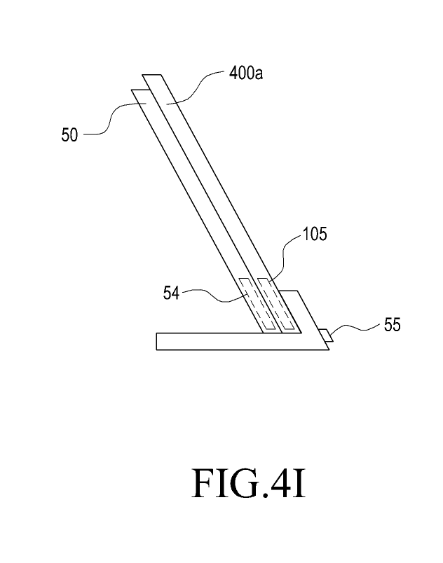

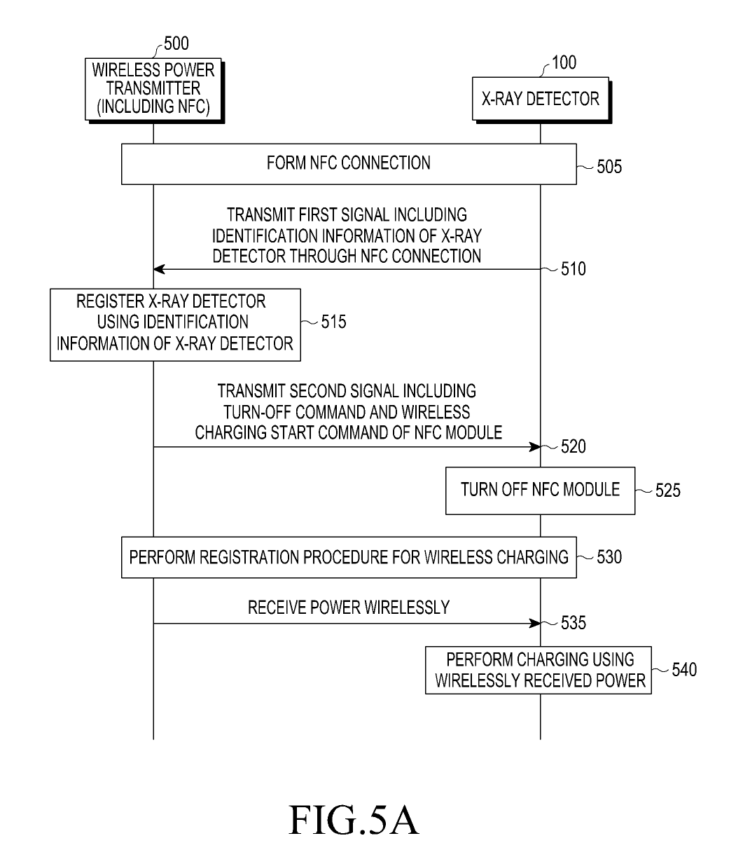

FIGS. 4A to 4I are conceptual diagrams illustrating a process in which an X-ray detector according to an exemplary embodiment is mounted on an imaging table, an imaging stand, or a workstation.

Referring to FIGS. 4A to 4D, the imaging table 20a may include a reception portion 22a, and an X-ray detector 100a may be seated in the reception portion 22a of the imaging table 20a. The imaging table 20a may include an integrated NFC and wireless charging module.

A module 23a in which a second wireless charging coil 23b and a second antenna 23c are integrated with each other may be mounted inside the reception portion 22a of the imaging table. The reception portion 22a may be provided with a button 25 formed on one surface thereof. The movement of current to the second wireless charging coil 23b may be stopped by an operation of pressing the button 25. Accordingly, it is possible to prevent an electromagnetic field 51 from being generated between the second wireless charging coil 23b and the first wireless charging coil 105 of the X-ray detector 100a.

The first antenna 106 of the X-ray detector 100a may transmit and receive a wireless signal S2 to and from a second antenna 23c of the reception portion 22a. The first antenna 106 of the X-ray detector 100a may receive identification information of the X-ray imaging apparatus through the wireless signal S2. The identification information may include an IP address, a serial number, a service set identifier (SSID), and the like of the X-ray imaging apparatus. However, the identification information is not limited thereto, and may correspond to various pieces of unique information of the X-ray imaging apparatus. The IP address may be address information for allowing the X-ray detector 100a to identify a transmission destination of data. The serial number is a number assigned when the X-ray imaging apparatus is manufactured, and may be a unique identification number of the X-ray imaging apparatus. The SSID is an identification number that is required in order for the X-ray detector 100a and the X-ray imaging apparatus to be connected to each other, and the X-ray imaging apparatus may have a unique SSID. For example, the SSID of the X-ray detector 100a may be changed through the wireless signal S2 so as to be the same as a unique SSID of the X-ray imaging apparatus.

The X-ray detector 100a may transmit and receive a wireless signal to and from the X-ray imaging apparatus, and then the button 25 may be pressed to supply current to the second wireless charging coil 23b. The second wireless charging coil 23b may generate the electromagnetic field S1 so as to generate current in the first wireless charging coil 105 of the X-ray detector 100a.

Referring to FIGS. 4E to 4G, the imaging stand 30a may include a case 31a and a reception portion 32a. The X-ray detector 100a may be seated in the reception portion 32a of the imaging stand 30a.

A module 33a in which a second wireless charging coil 33b and a second antenna 33c are integrated with each other may be mounted inside the reception portion 32a of the imaging table. The reception portion 32a may be provided with a button 35 formed on one surface thereof. The movement of current to the second wireless charging coil 33b may be stopped by an operation of pressing the button 35, so that it is possible to prevent an electromagnetic field from being generated between the second wireless charging coil 33b and the first wireless charging coil 105 of FIG. 4D of the X-ray detector 100a.

The first antenna 106 of FIG. 4D of the X-ray detector 100a may transmit and receive a wireless signal to and from the second antenna 33c of the reception portion 32a. The first antenna 106 of FIG. 4D of the X-ray detector 100a may receive identification information of the X-ray imaging apparatus through the wireless signal.

The X-ray detector 100a may transmit and receive a wireless signal to and from the X-ray imaging apparatus, and then the button 35 is pressed to supply current to the second wireless charging coil 33b. The second wireless charging coil 33b may generate an electromagnetic field to generate current in the first wireless charging coil 105 of FIG. 4D of the X-ray detector 100a.

Referring to FIGS. 4H and 4I, a cradle 50 of an X-ray detector is shown.

A module 53 in which a second wireless charging coil and an antenna are integrated with each other may be mounted inside the cradle 50. The cradle 50 may be provided with a button 55 formed on one surface thereof. The movement of current to the second wireless charging coil may be stopped by an operation of pressing the button 55, so that it is possible to prevent an electromagnetic field from being generated between the second wireless charging coil and the first wireless charging coil 105 of an X-ray detector 400a.

The second antenna 23c of the X-ray detector 400a may transmit and receive a wireless signal to and from a second antenna of the cradle 50. The second antenna 23c of the X-ray detector 400a may receive identification information of the workstation through the wireless signal.

The X-ray detector 400a may transmit and receive a wireless signal to and from the second antenna of the cradle, and then the button 55 may be pressed to supply current to the second wireless charging coil of the cradle 50. The second wireless charging coil of the cradle 50 may generate an electromagnetic field to generate current in the first wireless charging coil 105 of the X-ray detector 400a.

According to various embodiments of the present disclosure, the button 55 may generate a sound by the pressing operation thereof. A controller of the cradle 50 may cut off the current supplied to the wireless charging coil of the cradle 50 according to the generated sound.

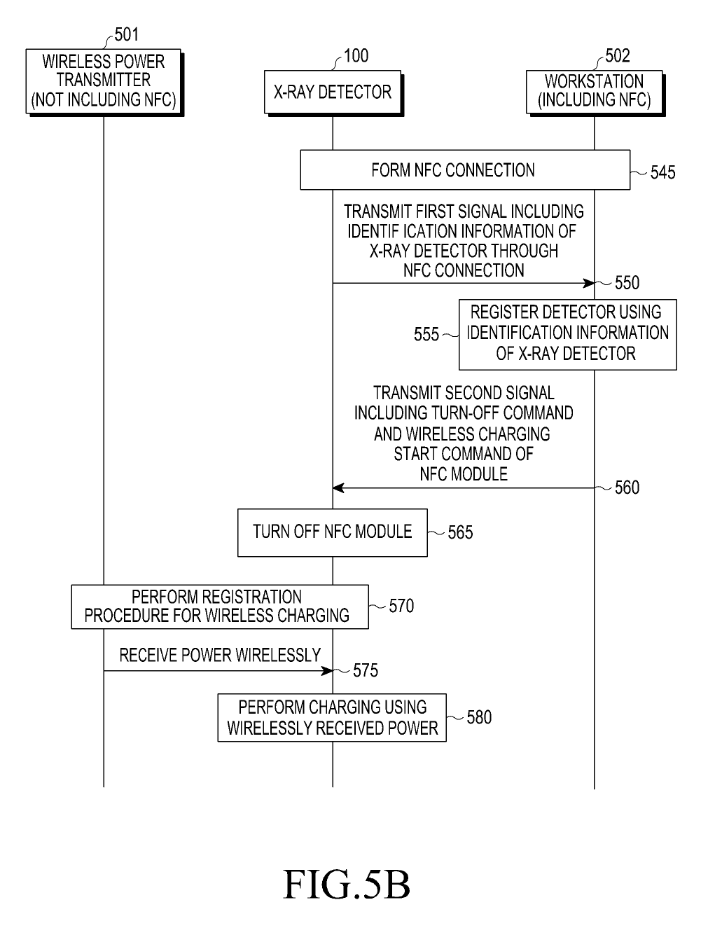

FIG. 5A is a flowchart illustrating the operation of a wireless power transmitter 500 and an X-ray detector 100 according to an exemplary embodiment. In the exemplary embodiment of FIG. 5A, it is assumed that a wireless power transmitter 500 includes an NFC module for NFC. The wireless power transmitter 500 may be included in at least one of the workstation 40, the imaging table 20, and the imaging stand 30.

In operation 505, the wireless power transmitter 500 may form an NFC connection with the X-ray detector 100. The X-ray detector 100 may include an NFC tag and may form an NFC connection, that is, an NFC pair, in an active communication mode or a passive communication mode.

In the case of operating in the active communication mode, the X-ray detector 100 may receive an initial command from the wireless power transmitter 500 through the NFC antenna. The X-ray detector 100 may transmit a response through the NFC antenna in response to the initial command using an internal power source. The response may include, for example, identification information defined in the NFC of the X-ray detector 100.

In the case of operating in the passive communication mode, the initial command may be received from the X-ray detector 100 via the NFC antenna. The X-ray detector 100 may modulate the initial command, that is, an RF signal, into power, and thereby form an NFC pair by transmitting a response command.

In operation 510, the X-ray detector 100 may transmit a first signal including the identification information of the X-ray detector 100 through the NFC connection.

In operation 515, the wireless power transmitter 500 may register the X-ray detector 100 using the identification information of the X-ray detector 100.

In operation 520, the wireless power transmitter 500 may transmit a second signal including a turn-off command and a wireless charging start command of the NFC module.

In operation 525, the X-ray detector 100 may turn off the NFC module.

In operation 530, the X-ray detector 100 may perform registration procedures for wireless charging. For example, the X-ray detector 100 may perform registration procedures defined in a WPC standard or an A4WP standard.

Upon completion of the registration procedures, in operation 535, the X-ray detector 100 may receive power wirelessly.

In operation 540, the X-ray detector 100 may perform charging using the wirelessly received power.

As described above, the X-ray detector 100 may turn off the NFC module before starting wireless charging. This is because when the wireless charging is started, a magnetic field or an electromagnetic field generated from the wireless power transmitter 500 may be introduced into the NFC module through an NFC antenna, and damage the NFC module. In an exemplary embodiment, the X-ray detector 100 may turn off the NFC module using the wireless charging start command as a trigger, even without a separate turn-off command, thereby preventing damage that may occur in the wireless charging process.

FIG. 5B is a flowchart illustrating the operations of the X-ray detector 100, a wireless power transmitter 501, and a workstation 502 according to another exemplary embodiment. In the exemplary embodiment of FIG. 5B, it is assumed that the wireless power transmitter 501 does not include the NFC module and that the workstation 502 includes the NFC module. For example, the wireless power transmitter 501 may be included in at least one of the imaging table 20 and the imaging stand 30.

In operation 545, the X-ray detector 100 may form an NFC connection with the workstation 502.

In operation 550, the X-ray detector 100 may transmit a first signal including identification information of the X-ray detector 100 through the NFC connection.

In operation 555, the workstation 502 may register the X-ray detector 100 using the identification information of the X-ray detector 100.

In operation 560, the workstation 502 may transmit a second signal including a turn-off command and a wireless charging start command of the NFC module to the X-ray detector 100.

In operation 565, the X-ray detector 100 may turn off the NFC module.

After turning off the NFC module, in operation 570, the X-ray detector 100 may perform registration procedures for wireless charging with the wireless power transmitter 501.

In operation 575, the wireless power transmitter 501 may receive power wirelessly.

In operation 580, the X-ray detector 100 may perform charging using the wirelessly received power.

In an exemplary embodiment, the X-ray detector 100 may turn off the NFC module before starting wireless charging using the wireless charging start command as a trigger, even without any separate turn-off command. Meanwhile, the X-ray detector 100 may be set to start wireless charging using completion of transmission of identification information via NFC as a trigger. The X-ray detector 100 may turn off the NFC module, as described above, before starting wireless charging.

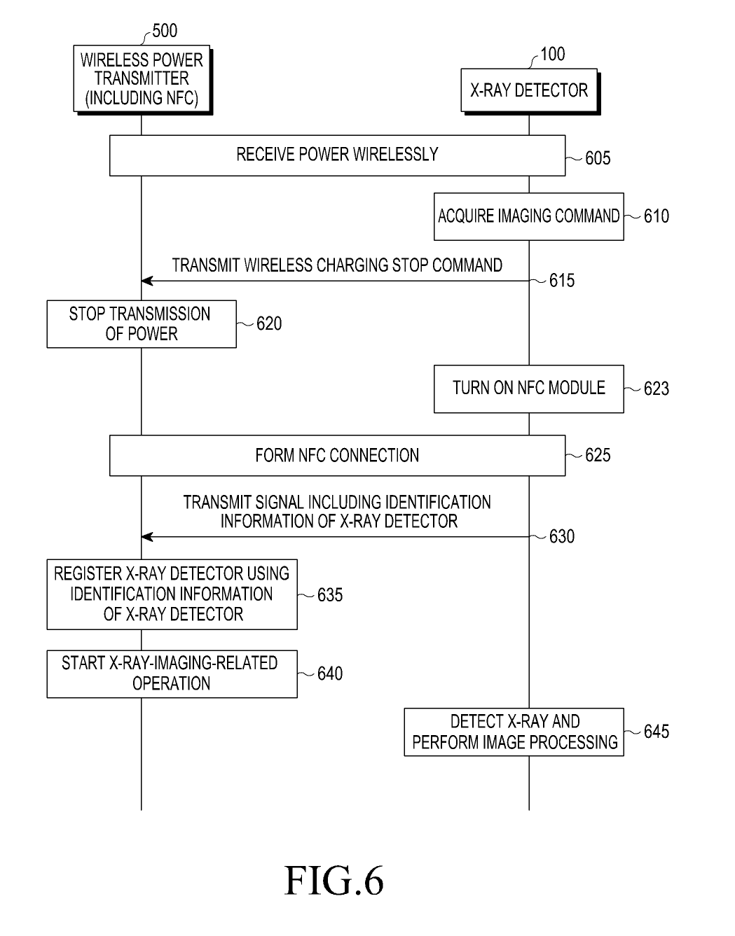

FIG. 6 is a flowchart illustrating the operations of a wireless power transmitter 500 and an X-ray detector 100 according to another exemplary embodiment. In the exemplary embodiment of FIG. 6, it is assumed that the wireless power transmitter 500 includes an NFC module. The wireless power transmitter 500 may be included in, for example, the imaging table 20, the imaging stand 30, or the workstation 40. In the exemplary embodiment of FIG. 6, a case where wireless charging is performed before the wireless power transmitter 500 forms NFC connection with the X-ray detector 100 will be described.

In operation 605, the X-ray detector 100 may receive power wirelessly. The X-ray detector 100 may receive power wirelessly after completing the registration procedures required by the wireless charging standard.

In operation 610, the X-ray detector 100 may acquire an imaging command. For example, the X-ray detector 100 may include the input device 190 of FIG. 3B. According to the operation of the input device 190, the X-ray detector 100 may acquire the imaging command. For example, the input device may be used for turning on/off the X-ray detector 100, or may be used for the imaging command.

When the imaging command is acquired, in operation 615, the X-ray detector 100 may transmit a wireless charging stop command to the wireless power transmitter 500. More specifically, the X-ray detector 100 may transmit the wireless charging stop command based on a communication scheme supported by the wireless charging standard. In another exemplary embodiment, the X-ray detector 100 may acquire an NFC registration command, and may transmit the wireless charging stop command using the acquisition of the NFC registration command as a trigger. That is, when the use of the NFC module such as imaging or NFC registration is required, the X-ray detector 100 may transmit the wireless charging stop command before turning on the NFC module.

In an exemplary embodiment, when the X-ray detector 100 complies with the WPC standard (or the Qi standard), the wireless charging stop command may be transmitted using an ON/OFF keying modulation/demodulation scheme by turning on/off a switch in a charging module. For example, the X-ray detector 100 may transmit the wireless charging stop command by transmitting an “End Power Transfer Packet (0x02)” to the wireless power transmitter 500. Table 1 shows the format of the “End Power Transfer Packet (0x02)” defined in the section 5.2.3.2 of the Qi standard (The Qi Wireless Power Transfer System Power Class 0 Specification V.1.2.2).

TABLE 1

| | | | | | | | | |
|---|---|---|---|---|---|---|---|---|
| B0 | b7 | b6 | b5 | b4 | b3 | b2 | b1 | b0 |

End Power Code

In the above, an end power code may indicate the cause of wireless charging termination, and the cause of wireless charging termination may be as shown in Table 2 below.

TABLE 2

| Reason | Value | Recommended usage of the values (informative) |
|---|---|---|
| Unknown | 0x00 | The Receiver may use this value if it does not have a specific |
| | | reason for terminating the power transfer or if none of the other |
| | | values listed in this table is appropriate. |
| Charge Complete | 0x01 | The Receiver should use this value if it determines that the |
| | | battery of the Mobile Device is fully charged. On receipt of an |
| | | End Power Transfer Packet containing this value, the Transmitter |
| | | should set any “charged” indication on its user interface that is |
| | | associated with the Receiver. |
| Internal Fault | 0x02 | The Receiver may use this value if it has encountered some |
| | | internal problem, e.g. a software or logic error. |
| Over Temperature | 0x03 | The Receiver should use this value if it has measured a |
| | | temperature within the Mobile Device that exceeds a limit. |
| Over Voltage | 0x04 | The Receiver should use this value if it has measured a voltage |
| | | within the Mobile Device that exceeds a limit. |
| Over Current | 0x05 | The Receiver should use this value if it has measured a current |
| | | within the Mobile Device that exceeds a limit. |
| Battery Failure | 0x06 | The Receiver should use this value if it has determined a problem |
| | | exists with the Mobile Device battery. |
| Reserved | 0x07 | The End Power Transfer Value = 0x07 (reconfigure) has been |
| | | deprecated, and should not be used. It may result in unpredictable |
| | | Power Transmitter behavior. |
| No Response | 0x08 | The Receiver should use this value if it determines that the |
| | | Transmitter does not respond to Control Error Packets as |
| | | expected (i.e. it does not increase or decrease its Primary Cell |
| | | current appropriately). |
| Reserved | 0x09 | — |
| Negotiation | 0x0A | A Power Receiver should use this value if it cannot negotiate a |
| Failure | | suitable Guaranteed Power level. |
| (Extended Power | | |
| Profile only) | | |
| Restarted Power | 0x0B | A Power Receiver should use this value if sees a need for |
| Transfer | | Foreign Object Detection with no power transfer in progress (see |
| (Extended Power | | Section 11.3. FOD based on quality factor change). To enable |
| Profile only) | | such detection, the power transfer has to be terminated. |
| | | Typically, the Power Transmitter then performs Foreign Object |
| | | Detection before restarting the power transfer. |
| Reserved | 0x0C to | — |
| | 0xFF | |

When an imaging command is input, the X-ray detector 100 may record, in the end power code, at least one of the reasons (e.g., Unknown, Charge Complete, Internal Fault, Over Temperature, Over Voltage, Over Current, Battery Failure, No Response, Negotiation Failure, and Restart Power Transfer) defined in the existing Qi standard, and may transmit a charging completion command to the wireless power transmitter 500. In another exemplary embodiment, the X-ray detector 100 may transmit the charging completion command to the wireless power transmitter 500 by recording the start of NFC as a reason using a reserved code.

In operation 620, the wireless power transmitter 500 may stop power transmission. The wireless power transmitter 500 may stop power transmission in response to the charging stop command, and may store and manage the reason therefor. Alternatively, the wireless power transmitter 500 may apply standby power in accordance with the stop of charging, or may not apply even the standby power for a predetermined time.

In another exemplary embodiment, when the X-ray detector 100 performs charging based on an A4WP standard (or an AFA standard) or an RF scheme, the charging stop command may be transmitted by transmitting a PRU Dynamic signal supported in BLE communication. The PRU Dynamic signal is a signal defined in, for example, 9.5.7 of AFA V.3.0. BSS, and may be used to report the status of the PRU. The PRU Dynamic may include emergency information (PRU alert), which is defined in 9.5.7.13 and may be as shown in Table 3 below.

TABLE 3

| 7 | 6 | 5 | 4 | 3 | 2 | 1 | 0 |
|---|---|---|---|---|---|---|---|
| Over- | Over- | Over-temp | PRU Self- | Charge | Wired | PRU | Adjust |
| voltage | current | | protection | Complete | Charger | Charge | Power |
| | | | | | Detect | Port | Response |

The X-ray detector 100 may transmit the PRU Dynamic by recording the reason for PRU self-protection in the emergency information (PRU alert). On the other hand, in the PRU Dynamic, there is no restriction on emergency reasons. The wireless power transmitter 500 that has received the PRU Dynamic including the PRU alert may stop the application of charging power to a resonator. The wireless power transmitter 500 may stop the application of charging power to the resonator, and may enter a latch fault mode or enter a local fault mode. Alternatively, the wireless power transmitter 500 may not apply even a beacon to the resonator for a predetermined time.

In operation 623, the X-ray detector 100 may turn on the NFC module.

In operation 625, the X-ray detector 100 may form an NFC connection with the wireless power transmitter 500.

In operation 630, the X-ray detector 100 may transmit a signal including identification information of the X-ray detector 100 to the wireless power transmitter 500.

In operation 635, the wireless power transmitter 500 may register the X-ray detector 100 using the identification information of the X-ray detector 100.

In operation 640, the wireless power transmitter 500 may initiate an X-ray-imaging-related operation. For example, the wireless power transmitter 500 may control the X-ray source 10 to apply an X-ray.

In operation 645, the X-ray detector 100 may detect the X-ray and perform image processing on the detected X-ray. The X-ray detector 100 may transmit an X-ray image to the wireless power transmitter 500 or the workstation 40. The X-ray detector 100 may transmit the X-ray image through another communication module (e.g., a data communication module), or may transmit the X-ray image through NFC.

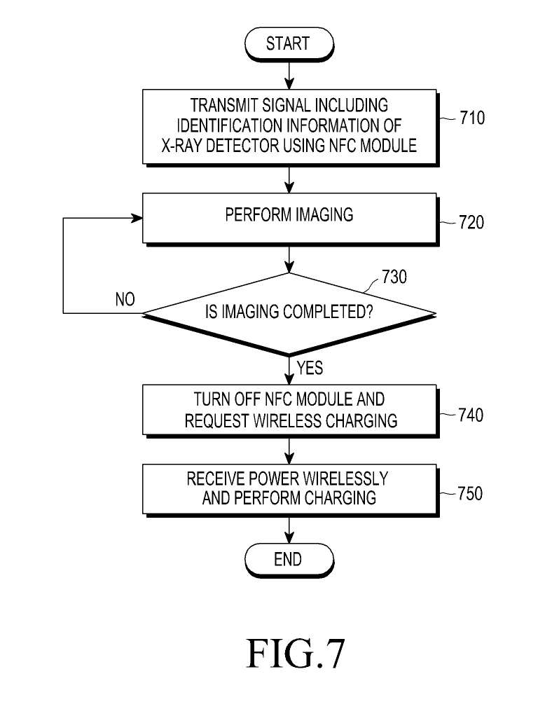

FIG. 7 is a flowchart illustrating the operation of an X-ray detector 100 according to an exemplary embodiment.

In operation 710, the X-ray detector 100 may transmit a signal including identification information of the X-ray detector 100 using an NFC module. That is, the X-ray detector 100 may perform registration in the workstation 40 through the identification information. As described above, the X-ray detector 100 may request stoppage of wireless charging from the wireless power transmitter 500 during an NFC-related operation. Accordingly, the signal transmission using the NFC module of operation 710 may be performed while wireless power reception is stopped.

In operation 720, the X-ray detector 100 may perform imaging.

In operation 730, the X-ray detector 100 may determine whether imaging is completed.

When it is determined that imaging has been completed, in operation 740, the X-ray detector 100 may turn off the NFC module and may request wireless charging. That is, the X-ray detector 100 may restart wireless charging using the completion of imaging as a trigger. The X-ray detector 100 may restart wireless charging by performing wireless charging registration procedures again, or may restart wireless charging by skipping at least some of the procedures.

In operation 750, the X-ray detector 100 may receive power wirelessly to perform charging. In another exemplary embodiment, the X-ray detector 100 may request wireless charging using NFC, using the completion of the registration procedures as a trigger.

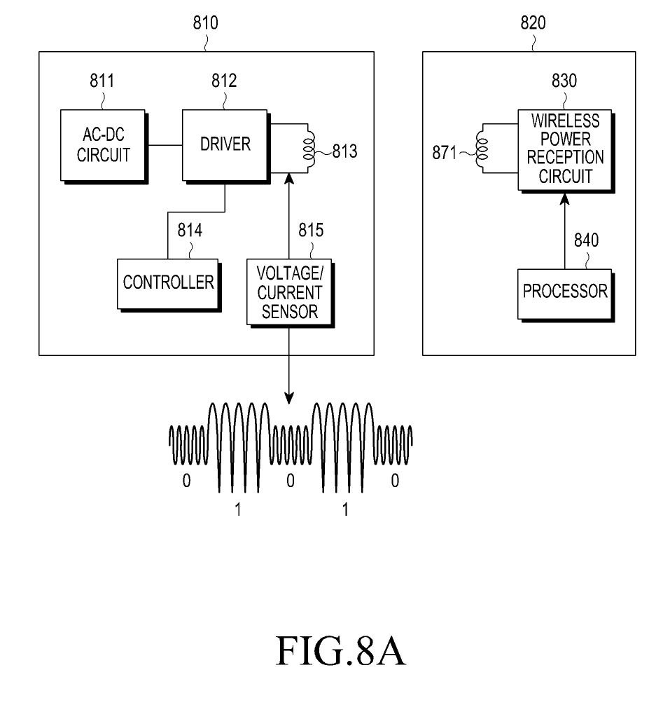

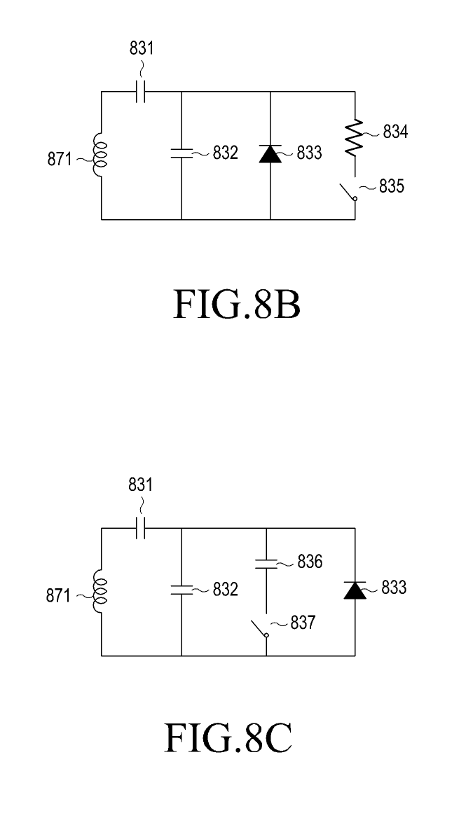

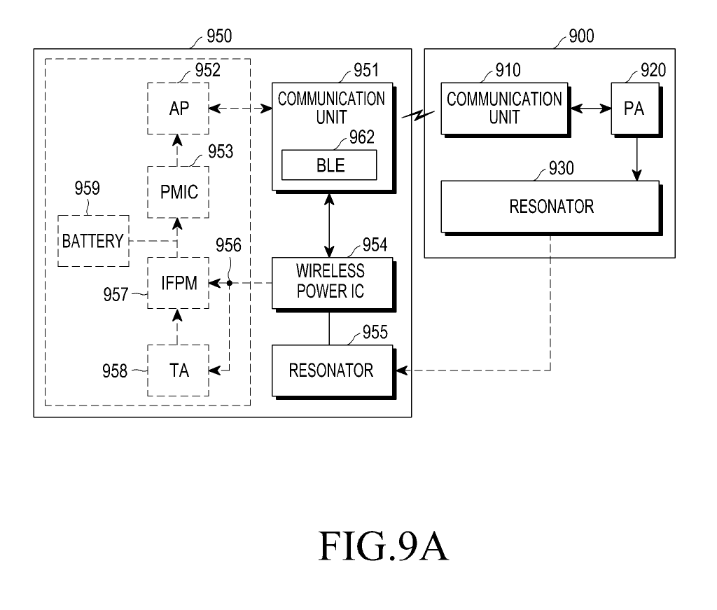

FIGS. 8A to 8C are conceptual diagrams illustrating a communication method in a WPC standard according to an exemplary embodiment. Referring to FIG. 8A, a wireless power transmitter 810 may include an AC-DC circuit 811, a driver 812, a coil 813, a controller 814, and a voltage/current sensor 815. An X-ray detector 820 may include a coil 871, a wireless power reception circuit 830, and a processor 840.

The AC-DC circuit 811 may convert power of a DC waveform into power of an AC waveform and may output the power of the AC waveform to the driver 812. The driver 812 may transmit input power to the coil 813. The coil 813 may wirelessly transmit the power to the coil 871 of the X-ray detector 820, that is, a secondary side coil. The wireless power reception circuit 830 may rectify the power of the AC waveform received from the coil 871 into power of a DC waveform, and perform converting or regulating. Alternatively, the wireless power reception circuit 830 may include a communication interface. For example, the wireless power reception circuit 830 may include a communication interface for in-band communication.

Referring to FIG. 8B, the communication interface according to an exemplary embodiment may include a resistor 834 and a switch 835 connected to a rectifier 833. Capacitors 831 and 832 may be connected to the coil 871, and the capacitors 831 and 832 and the coil 871 may constitute a resonant circuit having a resonant frequency set in, for example, the WPC standard. A processor 840 may control ON/OFF of the switch 835. The resistor 834 may be connected to the coil 871 when the switch 835 is turned on, and the resistor 834 may not be connected to the coil 871 when the switch 835 is turned off. The coil 813 and the coil 871 may be coupled to each other, so that an impedance facing the X-ray detector 820 in the coil 813 may be changed depending on whether the resistor 834 is connected to the coil 871. The switch 835 may be repeatedly turned on and off under the control of the processor 840. In this case, the result obtained by measuring a voltage or a current of the coil 813 using the voltage/current sensor 815 may be the same as changing relatively small amplitudes and relatively large amplitudes alternatively, as shown in the graph of FIG. 8A. The controller 814 may interpret information transmitted by the processor 840 by interpreting corresponding amplitudes.

Meanwhile, in the embodiment of FIG. 8C, the communication interface may also include a switch 837 and a capacitor 836. As described above, when the NFC-related operation of the X-ray detector 820 is requested or imaging is requested, the X-ray detector 820 may generate a signal corresponding to a charging stop command through ON/OFF operation of the switch 837. The wireless power transmitter 810 may detect the charging stop command based on sensing data of the voltage/current sensor 815, and thereby may stop the charging.

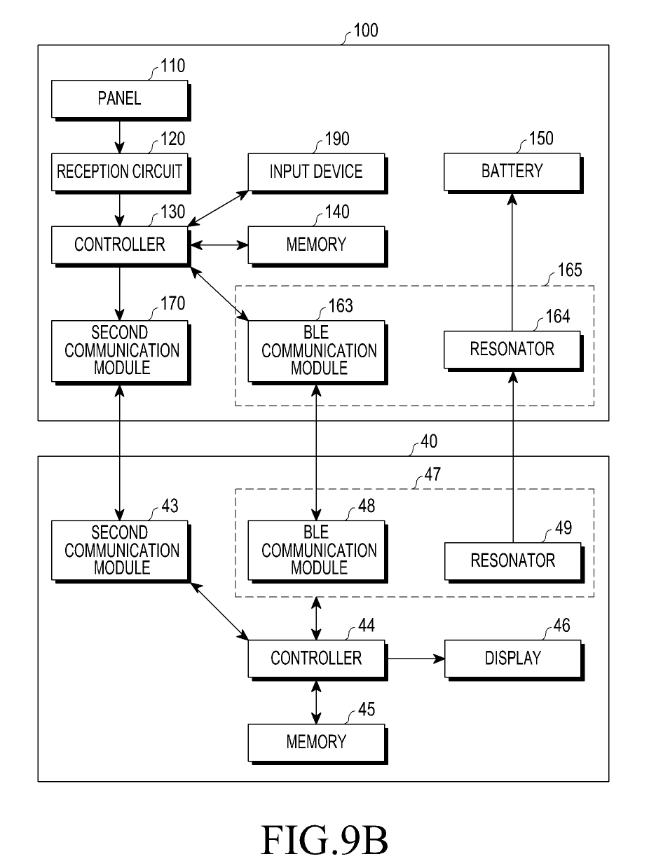

FIGS. 9A and 9B are block diagrams illustrating an X-ray detector 950 and a wireless power transmitter 900 based on an A4WP standard.

A wireless power transmitter 900 may include a communication unit 910, a power amplifier (PA) 920, and a resonator 930. An X-ray detector 950 may include a communication unit (WPT Communication IC) 951, an application processor (AP) 952, a power management integrated circuit (PMIC) 953, a wireless power integrated circuit (WPIC) 954, a resonator 955, an interface power management IC (IFPM) 957, a wired charging adapter (travel adapter (TA)) 958, and a battery 959.

The communication unit 951 may be implemented as a Wi-Fi/Bluetooth (BT) combo IC, and may perform communication with the communication unit 951 based on a predetermined method, for example, a BLE method. For example, the communication unit 951 of the X-ray detector 950 may transmit a PRU dynamic signal to a communication unit 910 of the wireless power transmitter 900. As described above, the PRU dynamic signal may include at least one of voltage information, current information, temperature information, and warning information of the X-ray detector 950.

Based on the received PRU dynamic signal, a power value output from the power amplifier 920 may be adjusted. For example, when an over-voltage, an over-current, or an over-temperature is applied to the X-ray detector 950, the power value output from the power amplifier 920 may be reduced. In addition, when the voltage or the current of the X-ray detector 950 is less than a predetermined value, the power value output from the power amplifier 920 may be increased.

Charging power from the resonator 930 may be wirelessly transmitted to the resonator 955.

The WPIC 954 may rectify the charging power received from the resonator 955, and may perform DC/DC conversion of the rectified power. The WPIC 954 may drive the communication unit 951 or charge the battery 959 using the converted power.

Meanwhile, a wired charging terminal may be inserted into the wired charging adapter 958. The wired charging terminal, such as a 30-pin connector or a USB connector, may be inserted into the wired charging adapter 958, and the battery 959 may be charged by receiving power supplied from an external power source.

The IFPM 957 may process the power applied from the wired charging terminal and may output the processed power to the battery 959 and the PMIC 953.

The PMIC 953 may manage wirelessly received power, wiredly received power, or power applied to each component of the X-ray detector 950. The AP 952 may receive power information from the PMIC 953, and may control the communication unit 951 to transmit a PRU dynamic signal for reporting the received power information. In various embodiments of the present disclosure, the PRU dynamic signal including a PRU alert may be transmitted in order to request stoppage of charging before performing an NFC-related operation or an imaging-related operation.

A node 956 connected to the WPIC 954 may also be coupled to the wired charging adapter 958. When a wired charging connector is inserted into the wired charging adapter 958, a predetermined voltage, for example, 5V may be applied to the node 956. The WPIC 954 may monitor the voltage applied to the node 956 to determine whether the wired charging connector is inserted into the wired charging adapter.

Meanwhile, the AP 952 has a stack corresponding to a predetermined communication scheme, for example, a Wi-Fi/BT/BLE stack. Accordingly, at the time of communication for wireless charging, the communication unit 951 may load the stack from the AP 952, and may then communicate with the communication unit 910 of the wireless power transmitter 900 using BT and BLE communication methods based on the stack.

However, there may occur the case in which data for performing wireless power transmission cannot be fetched from the AP 952 when the AP 952 is powered off or the case in which power is lost to such an extent that the AP 952 cannot be kept turned on while the data is fetched from a memory in the AP 952 and used.

In this way, when the remaining capacity of the battery 959 is below a minimum power threshold, the AP 952 may be turned off, and wireless charging may be performed using some components for wireless charging disposed inside the wireless power receiver, such as the communication unit 951, the WPIC 954, the resonator 955, and the like. Here, a state in which sufficient power to turn on the AP 952 cannot be supplied may be referred to as a dead-battery state.

In the dead-battery state, the communication unit 951 may not receive the stack of the predetermined communication scheme, for example, the Wi-Fi/BT/BLE stack, from the AP 952. In anticipation of this case, a part of the stack of the predetermined communication scheme, for example, a BLE stack, may be fetched from the AP 952 and may be stored in a memory 962 of the communication unit 951. Accordingly, the communication unit 951 may communicate with the wireless power transmitter 900 using the stack of the communication scheme stored in the memory 962, that is, a wireless charging protocol, for wireless charging. At this time, the communication unit 951 may have an internal memory. The BLE stack may be stored in a ROM-type memory in a stand alone (SA) mode.

As described above, a mode in which the communication unit 951 performs communication using the stack of the communication scheme stored in the memory 962 may be referred to as the SA mode. Accordingly, the communication unit 951 may manage charging procedures based on the BLE stack.

Referring to FIG. 9B, the X-ray detector 100 may include a wireless charging module 165, and the wireless charging module 165 may include a BLE communication module 163 and a resonator 164. In this exemplary embodiment, since BLE communication is provided, an NFC module may not be included in the X-ray detector 100. In this case, the X-ray detector 100 may include identification information of the X-ray detector 100 in a signal defined in the BLE communication and transmit the signal to the workstation 40. The workstation 40 may also include a wireless charging module 47 including a BLE communication module 48 and a resonator 49. The workstation 40 may register the X-ray detector 100 using the identification information included in the signal received via the BLE communication module 48. On the other hand, when imaging is requested, the X-ray detector 100 may transmit a PRU Dynamic signal requesting stoppage of charging via the BLE communication module 48, and the workstation 40 may also stop charging accordingly.

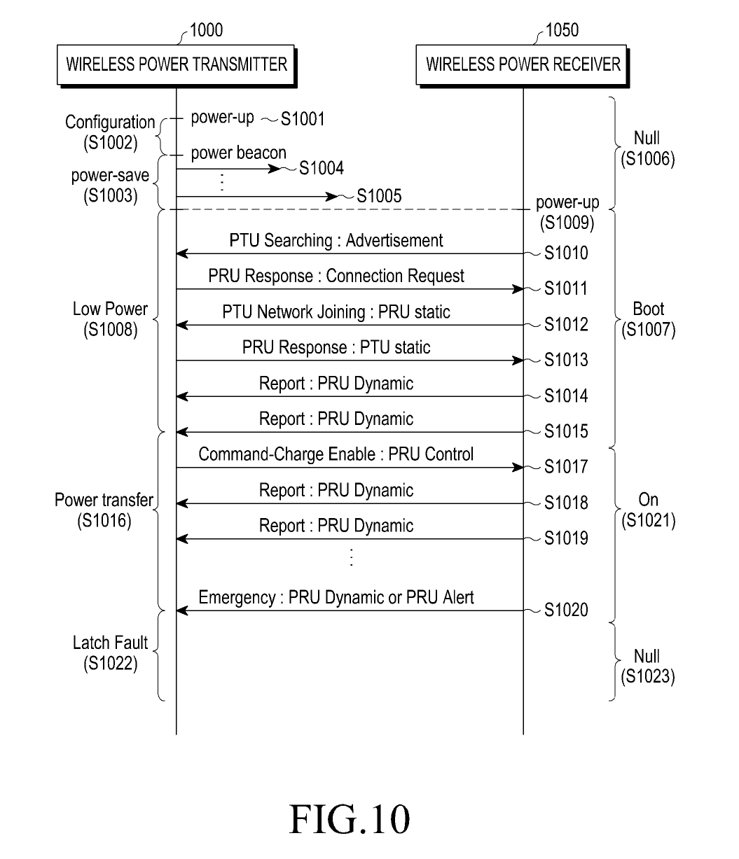

FIG. 10 is a flowchart illustrating the operations of a wireless power transmitter 1000 in accordance with an A4WP standard and a wireless power receiver 1050 included in an X-ray detector.

As shown in FIG. 10, in operation S1001, power may be applied to a wireless power transmitter 1000.

In operation S1002, when the power is applied, the wireless power transmitter 1000 may configure an environment.

In operation S1003, the wireless power transmitter 1000 may enter a power-saving mode. In the power-saving mode, the wireless power transmitter 1000 may apply each of different types of power beacons for detection, with their respective periods. For example, the wireless power transmitter 1000 may apply the power beacons (e.g., short beacons or long beacons) for detection in operation S1004 and S1005. The power beacons for detection in operations S1004 and S1005 may have different power values. One or both of the power beacons for detection in operations S1004 and S1005 may have sufficient power to drive the communication unit of a wireless power receiver 1050. For example, the wireless power receiver 1050 may communicate with the wireless power transmitter 1000 by driving its communication unit by means of one or both of the power beacons for detection in operation S1004 and S1005. At this time, this state may be referred to as a null state (S1006).

The wireless power transmitter 1000 may detect a load variation caused by disposition of the wireless power receiver 1050. The wireless power transmitter 1000 may enter a low-power mode in operation S1008. Meanwhile, the wireless power receiver 1050 may drive the communication unit with power received from the wireless power transmitter 1000 in operation S1009.

The wireless power receiver 1050 may transmit a wireless power transmitter searching (PTU searching) signal to the wireless power transmitter 1000 in operation S1010. The wireless power receiver 1050 may transmit the PTU searching signal as a BLE-based Advertisement (AD) signal. The wireless power receiver 1050 may transmit the PTU searching signal periodically until it receives a response signal from the wireless power transmitter 1000 or until after a predetermined time period has lapsed.

When the PTU searching signal is received from the wireless power receiver 1050, the wireless power transmitter 1000 may transmit a PRU response signal in operation S1011. Here, the PRU response signal may form a connection between the wireless power transmitter 1000 and the wireless power receiver 1050.

The wireless power receiver 1050 may transmit a PRU static signal in operation S1012. Here, the PRU static signal may indicate the state of the wireless power receiver 1050 and may request joining in a wireless power network managed by the wireless power transmitter 1000.

The wireless power transmitter 1000 may transmit a PTU static signal in operation S1013. The PTU static signal transmitted by the wireless power transmitter 1000 may indicate the capabilities of the wireless power transmitter 1000.

When the wireless power transmitter 1000 and the wireless power receiver 1050 transmit and receive the PRU static signal and the PTU static signal, the wireless power receiver 1050 may transmit a PRU dynamic signal periodically in operations S1014 and S1015. The PRU dynamic signal includes at least one piece of parameter information measured by the wireless power receiver 1050. For example, the PRU dynamic signal may include information about a voltage at the rear end of a rectifier of the wireless power receiver 1050. The state of the wireless power receiver 1050 during the operations S1009 through S1015 may be referred to as a boot state (S1007).

The wireless power transmitter 1000 may enter a power transfer mode in operation S1016. The wireless power transmitter 1000 may transmit a PRU control signal, that is, a signal for commanding the wireless power receiver 1050 to perform charging in operation S1017. In the power transfer mode, the wireless power transmitter 1000 may transmit charging power.

The PRU control signal transmitted by the wireless power transmitter 1000 may include information that enables/disables charging of the wireless power receiver 1050 and permission information. The PRU control signal may be transmitted each time a charged state is changed. For example, the PRU control signal may be transmitted, for example, every 250 ms, or may be transmitted in the event of a parameter change. The PRU control signal may be configured to be transmitted within a predetermined threshold time, for example, within 1 second, even though no parameter is changed.

The wireless power receiver 1050 may change a setting according to the PRU control signal and may transmit a wireless power receiver (PRU) dynamic signal to report the state of the wireless power receiver 1050 in operations S1018 and S1019. The PRU dynamic signal transmitted by the wireless power receiver 1050 may include information about at least one of a voltage, a current, a wireless power receiver state, and a temperature. The state of the wireless power receiver 1050 during the operations S1017 through S1020 may be referred to as an ON state (S1021).

Meanwhile, the wireless power receiver 1050 may sense an NFC-related operation start command or an imaging start command. The wireless power receiver 1050 may transmit a warning signal to the wireless power transmitter 1000 in operation S1020. The warning signal may be transmitted as a PRU Dynamic signal or as an alert signal. When the wireless power transmitter 1000 receives the warning signal, it may enter a latch fault mode in operation S1022. The wireless power receiver 1050 may enter a null state in operation S1023.

Meanwhile, the wireless power receiver 1050 may allow the identification information of the X-ray detector to be included in at least one of an advertisement signal, a PRU static signal, and a PRU dynamic signal. In this case, the X-ray detector may not include a separate NFC module.

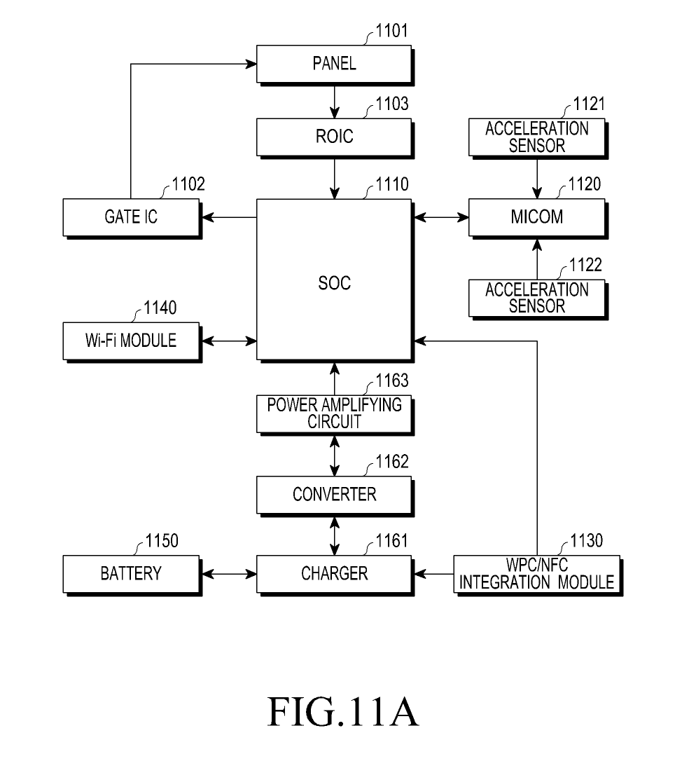

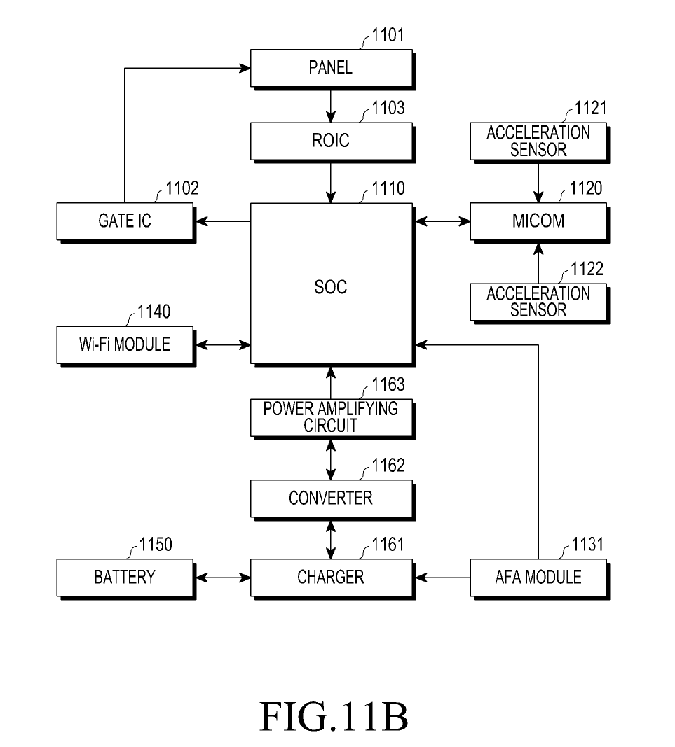

FIG. 11A is a block diagram illustrating an X-ray detector that performs wireless charging in accordance with a WPC standard according to an exemplary embodiment.

A system on a chip (SOC) 1110 is a main substrate, and may include, for example, a controller. A read out integrated chip (ROIC) 1103 may receive an electrical signal obtained by converting the X-ray of the panel 1101, process the received electrical signal, and provide the processed electrical signal to the SOC 1110. For example, the ROIC 1103 may include at least one of a switch and an ADC. A gate IC 1102 may select a row to be read out of the panel 1101 under the control of the SOC.

A MICOM 1120 may manage movement information, shock information, temperature information, and the like of the X-ray detector using sensing data from an acceleration sensor 1121 and a temperature sensor 1122, for example, in the state in which the SOC 1110 is turned off.

An WPC/NFC integration module 1130 may perform NFC with a workstation or the like, or may wirelessly receive power from the workstation or the like. As described above, the WPC/NFC integration module 1130 may request stoppage of wireless charging from the workstation or the like before NFC is performed or before imaging is started. When NFC is completed or imaging is completed, setting to initiate wireless charging may be made. The X-ray detector may include the WPC/NFC integration module 1130, a coil for receiving power wirelessly, a rectifier for processing received power, a converter, an antenna for NFC, and a communication module capable of generating an NFC signal.

A Wi-Fi module 1140 may wirelessly transmit an X-ray image generated by the SOC 1110 to another electronic device such as a workstation. In various embodiments of the present disclosure, the WPC/NFC integration module 1130 may also transmit the X-ray image to other electronic devices, and in this case, the Wi-Fi module 1140 may not be included in the X-ray detector.

The power received from the WPC/NFC integration module 1130 may be transmitted to a charger 1161, and the charger 1161 may charge the battery 1150 using the transmitted power. On the other hand, the charger 1161 may be configured to include a PMIC. In this case, the power from the battery 1150 may be supplied to a converter 1162 through the charger 1161, and the converter 1162 may process the supplied power suitable for each piece of hardware and provide the processed power to a power amplifying circuit 1163. The power amplifying circuit 1163 may amplify the received power and may provide the amplified power to various hardware such as the SOC 1110.

FIG. 11B is a block diagram illustrating an X-ray detector according to an exemplary embodiment. In contrast to FIG. 11A, the X-ray detector may include an AFA module 1131 in place of the WPC/NFC integration module 1130. The AFA module 1131 is based on the A4WP standard as described above, and may include a resonator for receiving power wirelessly, a rectifier for processing received power, a converter, and a BLE communication module.

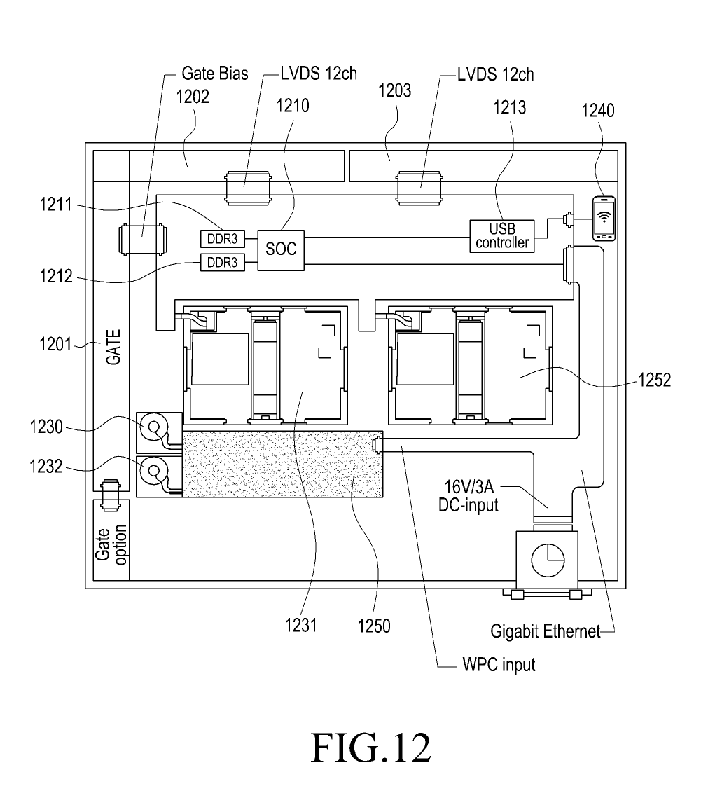

FIG. 12 is a circuit diagram illustrating an X-ray detector according to an exemplary embodiment.

An SOC 1210 may be disposed on a main board (MAIN B′d) and may input and output data with at least one RAM (DDR3) 1211 and 1212. In addition, the SOC 1210 may be connected to a USB controller 1213 so that the SOC 1210 may input/output data with a Wi-Fi module 1240. An X-ray image generated from the SOC 1210 may be transmitted to an external electronic device via the Wi-Fi module 1240. Meanwhile, the SOC 1210 may control a gate driver 1201 to select a row to be read out of a panel. The gate driver 1201 may be connected to the panel and may activate a photoelectric conversion element corresponding to the row to be read out of the panel. The photoelectric conversion element may convert an X-ray into an electrical signal and output the electrical signal to a first ROIC 1202 and a second ROIC 1203. The first ROIC 1202 and the second ROIC 1203 may convert the electrical signal into a digital signal and transmit the digital signal to the SOC 1210. The SOC 1210 may sequentially scan columns to be read out and may acquire electrical signals associated with the X-rays from all pixels of the entire panel. The SOC 1210 may generate an X-ray image using the electrical signal. Batteries 1231 and 1252 may be disposed below the main board (MAIN B′d). The WPC/NFC integration module 1250 may be disposed below the batteries 1231 and 1252 and may be coupled to coils and antennas 1230 and 1232.

While the present disclosure has been shown and described with reference to certain embodiments thereof, it will be understood by those skilled in the art that various changes in form and details may be made therein without departing from the spirit and scope of the present disclosure as defined by the appended claims.

What is claimed is:

1. An X-ray detector comprising:
a housing;
a panel positioned inside the housing, and configured to detect an X-ray and convert the detected X-ray into an electrical signal;
a controller configured to generate an X-ray image using the electrical signal; and
an integrated module comprising a wireless charging module for wirelessly receiving power from a wireless power transmitter and a Near Field Communication (NFC) module for performing NFC with an external electronic device.

2. The X-ray detector of claim 1, wherein the integrated module is configured to, when an NFC start command is acquired while the power is wirelessly received from the wireless power transmitter, stop reception of the power before performing the NFC, and turn on the NFC module.

3. The X-ray detector of claim 2, wherein the wireless charging module is configured to wirelessly receive the power based on a Wireless Power Consortium (WPC) standard, and request stoppage of transmission of the power from the wireless power transmitter based on an ON/OFF keying modulation/demodulation scheme defined in the WPC standard.

4. The X-ray detector of claim 3, wherein the wireless charging module is further configured to request the stoppage of transmission of the power from the wireless power transmitter, using an end power transfer packet defined in the WPC standard.

5. The X-ray detector of claim 2, wherein
the wireless charging module is further configured to wirelessly receive the power based on an Alliance for Wireless Power (A4WP) standard, and
the wireless charging module comprises a Bluetooth Low Energy (BLE) communication module that complies with the A4WP standard.

6. The X-ray detector of claim 5, wherein the wireless charging module is further configured to transmit a message for requesting stoppage of transmission of the power from the wireless power transmitter, through the BLE communication module.

7. The X-ray detector of claim 6, wherein the message for requesting the stoppage of transmission of the power is a PRU dynamic message defined in the A4WP standard.

8. The X-ray detector of claim 2, wherein the integrated module is further configured to:
transmit a signal including identification information of the X-ray detector through the NFC module,
turn off the NFC module when the signal including the identification information of the X-ray detector is completely transmitted, and
request transmission restart of the power from the wireless power transmitter.

9. The X-ray detector of claim 1, wherein the integrated module is configured to, when an imaging start command for the X-ray is acquired using the panel while the power is wirelessly received from the wireless power transmitter, request stoppage of reception of the power before performing the NFC, and turn on the NFC module.

10. The X-ray detector of claim 9, wherein the integrated module is further configured to, when imaging for the X-ray is completed using the panel, request restart of power transmission from the wireless power transmitter.

11. The X-ray detector of claim 1, wherein
the wireless charging module comprises a coil for receiving the power from the wireless power transmitter,
the NFC module comprises an antenna for transmitting and receiving a signal by the NFC, and
the antenna is disposed to surround the coil.

12. The X-ray detector of claim 1, further comprising:
a frame configured to be disposed between the panel and the integrated module.

13. The X-ray detector of claim 1, further comprising:
at least one circuit board configured to be electrically connected to the panel and a battery of the X-ray detector,
wherein the wireless charging module is disposed to be spaced apart from the at least one circuit board so as to avoid electromagnetic interference.

14. The X-ray detector of claim 13, wherein the battery is disposed between the wireless charging module and the at least one circuit board.

15. The X-ray detector of claim 13, further comprising:
a blocking member configured to be disposed between the at least one circuit board and the wireless charging module such that the blocking member blocks electromagnetism generated from the wireless charging module.

16. An X-ray detector comprising:
a housing;
a panel positioned inside the housing, and configured to detect an X-ray and convert the detected X-ray into an electrical signal;
a controller configured to generate an X-ray image using the electrical signal; and
an integrated module comprising a wireless charging module for wirelessly receiving power from a wireless power transmitter and a Bluetooth Low Energy (BLE) communication module for performing BLE communication with the wireless power transmitter,
wherein, when an imaging start command for the X-ray is acquired using the panel while the power is wirelessly received from the wireless power transmitter, the integrated module requests stoppage of transmission of the power from the wireless power transmitter.

17. The X-ray detector of claim 16, wherein the BLE communication module is configured to transmit a message for requesting the stoppage of transmission of the power from the wireless power transmitter.

18. The X-ray detector of claim 17, wherein the message for requesting the stoppage of transmission of the power is a PRU dynamic message defined in an Alliance for Wireless Power (A4WP) standard.

19. The X-ray detector of claim 16, wherein the integrated module is configured to, when imaging for the X-ray is completed using the panel, request restart of power transmission from the wireless power transmitter.

20. An X-ray imaging apparatus comprising:
an X-ray output device configured to apply an X-ray;
an X-ray detector comprising a sensing unit for detecting the X-ray, a battery, a wireless charging module electrically connected to the battery to generate current using a change in an external magnetic field, and an Near Field Communication (NFC) module for performing NFC with an external electronic device; and
a reception portion configured to receive the X-ray detector.
